(19) United States
(12) Patent Application Publication
(10) Pub. No.: US 2012/0034192 A1
(43) Pub. Date: Feb. 9, 2012

(54) COMPOSITIONS AND METHODS FOR ENHANCING CELL REPROGRAMMING

(76) Inventors: Richard A. Young, Weston, MA (US); Steve Bilodeau, Cambridge, MA (US); Michael H. Kagey, Somerville, MA (US)

(21) Appl. No.: 13/119,891

(22) PCT Filed: Sep. 21, 2009
(86) PCT No.: PCT/US2009/057692

§ 371 Date: Oct. 3, 2011

Related U.S. Application Data
(60) Provisional application No. 61/098,327, filed on Sep. 19, 2008.

(51) Int. Cl. A61K 35/12; C40B 30/06; C12N 5/10; C12N 5/071; C12Q 1/68

(52) U.S. Cl. 424/93.7; 435/375; 435/377; 435/366; 435/6.1; 435/6.13; 506/10

(57) ABSTRACT

The invention provides compositions and methods of use to enhance reprogramming of mammalian cells. Certain compositions and methods of the invention are of use to enhance generation of induced pluripotent stem cells by reprogramming somatic cells. Certain compositions and methods of the invention are of use to enhance reprogramming of pluripotent mammalian cells to a differentiated cell type. Certain compositions and methods of the invention are of use to enhance reprogramming of differentiated mammalian cells of a first cell type to differentiated mammalian cells of a second differentiated cell type. The reprogrammed somatic cells are useful for a number of purposes, including treating or preventing a medical condition in an individual. The invention further provides methods for identifying an agent that enhances or contributes to reprogramming mammalian cells. Certain of the inventive compositions and methods relate to inhibiting histone methylation.

### RELATED APPLICATIONS

This application claims priority to, and the benefit of, U.S. Provisional Application No. 61/098,327, filed Sep. 19, 2008. The entire contents of the afore-mentioned applications are incorporated herein by reference.

### GOVERNMENTAL FUNDING

The invention described herein was supported, in whole or in part, by grant HG002668 from the National Institutes of Health. The United States government has certain rights in the invention.

### BACKGROUND OF THE INVENTION

Stem cells are cells that are capable of self-renewal and of giving rise to more differentiated cells. Embryonic stem (ES) cells, for example, which can be derived from the inner cell mass of a normal embryo in the blastocyst stage, can differentiate into the multiple specialized cell types that collectively comprise the body (See, e.g., U.S. Pat. Nos. 5,843,780 and 6,200,806, Thompson, J. A. et al. Science, 282:1145-7, 1998). As cells differentiate they undergo a progressive loss of developmental potential that has generally been considered largely irreversible. Somatic cell nuclear transfer (SCNT) experiments, however, showed that nuclei from differentiated adult cells could be reprogrammed to a totipotent state by factors present in the oocyte cytoplasm.

In addition to being of immense scientific interest, human cells with the property of pluripotency hold great clinical promise for applications in regenerative medicine such as cell/tissue replacement therapies for disease. However, SCNT and conventional methods of obtaining ES cells suffer from a number of limitations that hamper their use in regenerative medicine applications, and alternatives have been avidly sought. Examples can be found in the scientific literature in which differentiated cells of a particular type have been converted into cells of a different type without apparently being reverted to a fully pluripotent state as an intermediate step. For example, dermal fibroblasts can be converted into muscle-like cells by forced expression of MyoD. However, such examples do not provide a general approach to generating large numbers of patient-specific cells of numerous diverse types.

In 2006 it was shown that cell lines with some of the properties of ES cells could be produced by introducing genes encoding four transcription factors associated with pluripotency, i.e., Oct3/4, Sox2, c-Myc and Klf4, into mouse skin fibroblasts via retroviral infection, and then selecting cells that expressed a marker of pluripotency, Fbx15, in response to these factors (Takahashi, K. & Yamanaka, S. Cell 126, 663-676, 2006). However, the resulting cells differed from ES cells in their gene expression and DNA methylation patterns and when injected into normal mouse blastocysts did not result in live chimeras. Subsequent work resulted in derivation of stable reprogrammed cell lines that, based on reported transcriptional, imprinting, and chromatin-modification profiles, appeared essentially identical to ES cells (Okita, K., et al., 448, 313-317, 2007; Wernig, M. et al. Nature 448, 318-324, 2007; Maherali, N. et al. Cell Stem Cell 1, 55-70, 2007). Subsequently it was shown that human somatic cells can also be reprogrammed to pluripotency using these factors. Furthermore, it was demonstrated that the combination of Oct4, Nanog, Sox2, and Lin28 was also able to reprogram somatic cells to a pluripotent state in vitro (Yu J, Science, 318(5858):1917-20, 2007). However, generating these cells also involved engineering the cells to express multiple transcription factors using retroviral transduction and occurs only with low efficiency.

There exists a need in the art for alternative and improved methods for reprogramming mammalian cells

### SUMMARY OF THE INVENTION

The present invention provides compositions and methods for reprogramming mammalian cells. In certain embodiments the compositions and methods are of use to reprogram somatic cells to a less differentiated cell state. In certain embodiments the compositions and methods are of use to reprogram somatic cells to pluripotent, embryonic stem cell-like cells, referred to herein as “ES-like cells” or “induced pluripotent stem cells (“iPS cells”). In certain embodiments the compositions and methods are of use to reprogram pluripotent cells to a more differentiated state. In certain embodiments the compositions and methods are of use to reprogram pluripotent cells to a desired differentiated cell type. In certain embodiments the compositions and methods are of use to reprogram mammalian cells from a first differentiated cell type to a second differentiated cell type. In certain embodiments such reprogramming does not require the generation of pluripotent cells as an intermediate step.

The invention provides methods of identifying pluripotency regulators such as genes and gene products that regulate pluripotency (e.g., whose expression promotes pluripotency or differentiation). The invention further provides pluripotency regulators identified using the inventive methods.

In one aspect, the invention provides a method of enhancing the reprogramming of mammalian cells comprising: (a) contacting mammalian cells with an agent that inhibits histone methylation; and (b) subjecting the cells to a reprogramming protocol so that at least some cells become reprogrammed to a desired cell state, wherein the agent enhances such reprogramming.

In certain embodiments of the invention the agent inhibits H3K9 methylation. In certain embodiments of the invention the agent inhibits histone methyltransferase activity. In certain embodiments of the invention the agent inhibits expression of a histone methyltransferase. In certain embodiments of the invention the histone methyltransferase is an H3K9 methyltransferase. In certain embodiments of the invention the histone methyltransferase is Suv39h1. In certain embodiments of the invention the histone methyltransferase is Suv39h2. In certain embodiments of the invention the histone methyltransferase is Ehmt1. In certain embodiments of the invention the histone methyltransferase is SetDB1. In certain embodiments at least two H3K9 methyltransferases (e.g., 2, 3, 4, etc.) are inhibited. In certain embodiments of the invention both Suv39h1 and Suv39h2 are inhibited. In certain embodiments of the invention the agent is an siRNA or shRNA that inhibits expression of a histone methyltransferase, e.g., an H3K9 methyltransferase, e.g., Suv39h1, Suv39h2, or SetDB1. In certain embodiments of the invention, the cells are differentiated cells, and reprogramming the cells comprises reprogramming the cells to a pluripotent state. In certain embodiments of the invention, the cells are iPS cells, and reprogramming the iPS cells comprises reprogramming the iPS cells to a desired cell type. In certain embodiments of the invention, the cells are differentiated cells of a first cell type, and the reprogramming protocol reprograms the cells to a second differentiated cell type. In certain embodiments of the invention, reprogramming efficiency is increased by at least a factor of 2. In certain embodiments of the invention, the cells are human cells. In certain embodiments of the invention, contacting the cells with the agent comprises culturing the cells in culture medium containing the agent. In certain embodiments of the invention, the cells are contacted with the agent for a limited period of time, e.g., 1-3, 1-5, 1-10, 3-5, 5-10, 10-20, or 20-30 days. In certain embodiments of the invention, the cells are modified to contain at least one reprogramming factor at levels greater than normally present in cells of that type. In certain embodiments of the invention, the cells comprise a nucleic acid construct that encodes the reprogramming factor, wherein the construct is not integrated into the cell genome. In certain embodiments of the invention, the cells are not genetically modified. In certain embodiments of the invention, the cells are not genetically modified to express c-Myc. In certain embodiments of the invention, the method further comprises assessing whether the cells have become reprogrammed to the desired cell state. In certain embodiments of the invention, the method further comprises separating cells that are reprogrammed to a desired state from cells that are not reprogrammed to a desired state. In certain embodiments of the invention, the method further comprises administering the reprogrammed cells to a subject.

The invention further provides a method comprising: (i) reprogramming somatic cells to a pluripotent state by a method comprising (a) contacting mammalian cells with an agent that inhibits histone methylation, and (b) subjecting the cells to a reprogramming protocol so that at least some cells become reprogrammed to a desired cell state, wherein the agent enhances such reprogramming; and (ii) reprogramming the pluripotent cells to a desired, differentiated cell type.

The invention further provides a method comprising: (i) reprogramming somatic cells to a pluripotent state; and (ii) reprogramming the pluripotent cells to a desired, differentiated cell type by a method comprising (a) contacting mammalian cells with an agent that inhibits histone methylation, and (b) subjecting the cells to a reprogramming protocol so that at least some cells become reprogrammed to a desired cell state, wherein the agent enhances such reprogramming.

The invention further provides a method comprising: (i) reprogramming somatic cells to a pluripotent state; and (ii) reprogramming the pluripotent cells to a desired, differentiated cell type, wherein step (i) and step (ii) are performed by a method comprising (a) contacting mammalian cells with an agent that inhibits histone methylation, and (b) subjecting the cells to a reprogramming protocol so that at least some cells become reprogrammed to a desired cell state, wherein the agent enhances such reprogramming.

In some embodiments of the inventive methods, the reprogramming protocol comprises inducing expression of at least one reprogramming factor in the cells.

The invention further provides a method of treating an individual in need thereof comprising: (i) obtaining somatic cells from the individual; (ii) reprogramming at least some of the somatic cells according to a method comprising (a) contacting mammalian cells with an agent that inhibits histone methylation, and (b) subjecting the cells to a reprogramming protocol so that at least some cells become reprogrammed to a desired cell state, wherein the agent enhances such reprogramming; and (iii) administering at least some of the reprogrammed cells to the individual. In some embodiments the method further comprises separating cells that are reprogrammed to a desired state from cells that are not reprogrammed to a desired state. The invention further provides a method of preparing a therapeutic composition comprising: (i) obtaining somatic cells from an individual suffering from a disorder in which cell therapy is indicated; (ii) reprogramming at least some of the somatic cells according to a method comprising (a) contacting mammalian cells with an agent that inhibits histone methylation, and (b) subjecting the cells to a reprogramming protocol so that at least some cells become reprogrammed to a desired cell state, wherein the agent enhances such reprogramming.

The invention further provides a composition comprising (i) a non-pluripotent somatic mammalian cell that comprises an introduced reprogramming factor; and (ii) an agent that inhibits histone methylation. In some embodiments the reprogramming factor is Oct4. In some embodiments the agent is an siRNA. In some embodiments the somatic cell is not genetically modified. In some embodiments the somatic cell does not contain exogenously introduced c-Myc at levels greater than normally present in somatic cells of that type. In some embodiments the cell is obtained from an individual suffering from a disorder for which cell therapy is indicated.

The invention further provides a composition comprising (i) an iPS cell; and (ii) an agent that inhibits histone methylation. In some embodiments the agent is an siRNA. In some embodiments the iPS cell is not genetically modified. In some embodiments the iPS cell is obtained by reprogramming a somatic cell obtained from an individual suffering from a disorder for which cell therapy is indicated.

The invention further provides a method of identifying an agent useful for modulating the reprogramming of mammalian cells comprising: (a) maintaining mammalian cells in culture in the presence of a candidate agent under conditions in which histone methylation is inhibited in the cells, wherein the mammalian cells are cells of a first cell type; and (b) determining, after a suitable time period, whether cells having one or more characteristics of a second cell type different from the first cell type are present in the culture, wherein the candidate agent is identified as being useful for modulating the reprogramming of mammalian cells if cells or cell colonies having one or more characteristics of the second cell type are present in amounts different than would be expected had the cells of the first cell type been cultured under identical conditions in the absence of the candidate agent. In some embodiments the cells of the first cell type are somatic cells. In some embodiments the cells of the first cell type are somatic cells and cells of the second cell type are ES cells. In some embodiments the cells of the first cell type are terminally differentiated cells. In some embodiments the cells of the first cell type are ES cells. In some embodiments the cells of the first cell type are iPS cells. In some embodiments the cells of the first cell type are iPS cells and cells of the second cell type are terminally differentiated cells. In some embodiments the cells contain at least one introduced reprogramming factor. In some embodiments the candidate agent is a small molecule. In some embodiments H3K9 methylation is inhibited. In some embodiments histone methylation is inhibited by contacting the cells with an siRNA that inhibits expression of a histone methyltransferase. In some embodiments cells of the first cell type are non-pluripotent somatic cells, cells of the second cell type are pluripotent cells, wherein the candidate agent is identified as being useful for enhancing the reprogramming of non-pluripotent mammalian somatic cells to a pluripotent state if cells or cell colonies having one or more characteristics of ES cells or ES cell colonies are present at levels greater than would be expected had the cells been cultured under identical conditions in the absence of the candidate agent.

The invention further provides a method of identifying an agent useful for modulating the reprogramming of mammalian cells comprising: (a) maintaining mammalian ES or iPS cells in culture in the presence of a candidate agent; and (b) assessing expression of an endogenous pluripotency gene by the cells, wherein the agent is identified as useful for modulating the reprogramming of mammalian cells if expression of the endogenous pluripotency gene is increased or decreased relative to the level of expression of said gene that would exist in the absence of the candidate agent. In some embodiments the agent is identified as useful for reprogramming mammalian somatic cells to a less differentiated state if expression is increased. In some embodiments the agent is identified as useful for reprogramming mammalian somatic cells to a more differentiated state if expression is decreased. In some embodiments the pluripotency gene is Oct4.

The invention further provides a method of identifying a gene whose inhibition modulates the reprogramming of mammalian cells comprising: (a) providing mammalian ES or iPS cells in culture; and (b) inhibiting expression of an endogenous candidate gene by the ES or iPS cells; and (c) assessing expression of an endogenous pluripotency gene by the cells, wherein the endogenous candidate gene is identified as one whose inhibition modulates the reprogramming of mammalian cells if expression of the endogenous pluripotency gene is increased or decreased relative to the level of expression of said gene that would exist in ES or iPS cells in which expression of the candidate gene is not inhibited. In some embodiments the gene is identified as one whose inhibition promotes reprogramming of mammalian somatic cells to a less differentiated state if expression of the endogenous pluripotency gene is increased. In some embodiments the gene is identified as one whose inhibition promotes reprogramming of mammalian cells to a more differentiated state if expression of the endogenous pluripotency gene is decreased. In some embodiments the pluripotency gene is Oct4. In some embodiments expression of the endogenous candidate gene is inhibited by RNAi.

The invention further provides a method of identifying an agent useful for modulating reprogramming of mammalian cells, the method comprising identifying an agent that inhibits expression or activity of a gene identified according to the method of gene identification described above.

The invention also provides methods for identifying an agent of use to reprogram somatic cells and/or that contributes to such reprogramming in combination with one or more other agents.

As noted herein, the present invention provides methods for treating a condition in an individual in need of treatment for a condition. In certain embodiments, somatic cells are obtained from the individual and reprogrammed using compositions and/or methods of the invention. It will be understood that the phrase “obtained from an individual” is used in a broad sense and encompasses situations in which the physical procedure of obtaining a tissue sample or blood sample from the individual is performed by the same individual or entity who performs the reprogramming and situations in which a third party (e.g., a health care provider) takes a tissue or blood sample from the individual), who then provides the sample (or cells from the sample) to the individual or entity that will perform the reprogramming. Thus, “obtaining” can mean “receiving from a third party”. Furthermore, “administering” can refer to physically administering or providing to a third party (e.g., a health care provider) for purposes of administration.

The reprogrammed cells may be expanded in culture. In some embodiments, pluripotent reprogrammed cells (which refers to the original reprogrammed cells and/or their progeny that retain the property of pluripotency) are maintained under conditions suitable for the cells to develop into cells of a desired cell type or cell lineage. In some embodiments, the cells are differentiated in vitro using protocols known in the art. The reprogrammed cells of a desired cell type are introduced into the individual to treat the condition. In certain embodiments, somatic cells obtained from the individual contain a mutation in one or more genes. In these instances, in certain embodiments the somatic cells obtained from the individual are first treated to repair or compensate for the defect, e.g., by introducing one or more wild type copies of the gene(s) into the cells such that the resulting cells express the wild type version of the gene. The cells are then reprogrammed and introduced into the individual. Alternately, the cells are reprogrammed and then treated to repair or compensate for the defect.

In certain embodiments, the somatic cells obtained from the individual are engineered to express one or more genes after being removed from the individual. The cells may be engineered by introducing a gene or expression cassette comprising a gene into the cells. The introduced gene may be one that is useful for purposes of identifying, selecting, and/or generating a reprogrammed cell. In certain embodiments the introduced gene(s) contribute to initiating and/or maintaining the reprogrammed state. In certain embodiments the expression product(s) of the introduced gene(s) contribute to producing the reprogrammed state but are dispensable for maintaining the reprogrammed state.

In certain other embodiments, methods of the invention can be used to treat individuals in need of a functional organ. In the methods, somatic cells are obtained from an individual in need of a functional organ, and reprogrammed by the methods of the invention to produce reprogrammed somatic cells. Such reprogrammed somatic cells are then cultured under conditions suitable for development of the reprogrammed somatic cells into a desired organ, which is then introduced into the individual.

It is contemplated that all embodiments described herein are applicable to the various aspects of the invention. It is also contemplated that the various embodiments of the invention and elements thereof can be combined with one or more other such embodiments and/or elements whenever appropriate.

### BRIEF DESCRIPTION OF THE DRAWINGS

FIG. 1: Overview of screening protocol for identification of pluripotency regulators in ES cells. Mouse Embryonic stem cells were seeded onto gelatin coated 384 well plates at a density of ˜2000 cells/well. Cells were infected on the following day with lentiviral vectors encoding shRNAs targeting selected chromatin factors, 24 hours post-infection cells were treated with puromycin to select for stably integrated virus. 5 days post-infection cell were fixed and stained with Hoechst dye (to identify nuclei) and for Oct4, Images were acquired with a Cellomics ArrayScan and analyzed to determine the Oct4 staining intensity.

FIG. 2: Positive controls for screen to identify pluripotency regulators in ES cells. Lentiviral shRNAs targeting Oct4 and Stat3 (a protein required for maintaining pluripotency) result in a decrease in Oct4 staining relative to the negative control infection with lentivirus encoding shRNA targeted to GFP. Inhibiting Tcf3 (a protein that primes cells for differentiation by repressing Oct4) expression results in an increase in Oct4 staining.

FIG. 3: Inhibiting histone methyltransferases modulates reprogramming efficiency.

FIG. 4: Effect of inhibiting H3K9 methyltransferases on reprogramming efficiency.

FIG. 5. Table 1 shows results of the screen to identify modulators of Oct4 expression. Genes whose inhibition resulted in an increase or decrease in Oct4 staining are categorized based on function and/or presence in various protein complexes.

### DETAILED DESCRIPTION OF THE INVENTION

### Definitions

“Agent” as used herein means any compound or substance such as, but not limited to, a small molecule, nucleic acid, polypeptide, peptide, drug, ion, etc.

“Exogenous” refers to a substance present in a cell or organism other than its native source. For example, the terms “exogenous nucleic acid” or “exogenous protein” refer to a nucleic acid or protein that has been introduced by a process involving the hand of man into a biological system such as a cell or organism in which it is not normally found or in which it is found in lower amounts. A substance will be considered exogenous if it is introduced into a cell or an ancestor of the cell that inherits the substance. In contrast, the term “endogenous” refers to a substance that is native to the biological system.

“Expression” refers to the cellular processes involved in producing RNA and proteins as applicable, for example, transcription, translation, folding, modification and processing. “Expression products” include RNA transcribed from a gene and polypeptides obtained by translation of mRNA transcribed from a gene.

A “genetically modified” or “engineered” cell refers to a cell into which an exogenous nucleic acid has been introduced by a process involving the hand of man (or a descendant of such a cell that has inherited at least a portion of the nucleic acid). The nucleic acid may for example contain a sequence that is exogenous to the cell, it may contain native sequences (i.e., sequences naturally found in the cells) but in a non-naturally occurring arrangement (e.g., a coding region linked to a promoter from a different gene), or altered versions of native sequences, etc. The process of transferring the nucleic into the cell can be achieved by any suitable technique. Suitable techniques include calcium phosphate or lipid-mediated transfection, electroporation, and transduction or infection using a viral vector. In some embodiments the polynucleotide or a portion thereof is integrated into the genome of the cell. The nucleic acid may have subsequently been removed or excised from the genome, provided that such removal or excision results in a detectable alteration in the cell relative to an unmodified but otherwise equivalent cell.

“Identity” refers to the extent to which the sequence of two or more nucleic acids or polypeptides is the same. The percent identity between a sequence of interest and a second sequence over a window of evaluation, e.g., over the length of the sequence of interest, may be computed by aligning the sequences, determining the number of residues (nucleotides or amino acids) within the window of evaluation that are opposite an identical residue allowing the introduction of gaps to maximize identity, dividing by the total number of residues of the sequence of interest or the second sequence (whichever is greater) that fall within the window, and multiplying by 100. When computing the number of identical residues needed to achieve a particular percent identity, fractions are to be rounded to the nearest whole number. Percent identity can be calculated with the use of a variety of computer programs known in the art. For example, computer programs such as BLAST2, BLASTN, BLASTP, Gapped BLAST, etc., generate alignments and provide percent identity between sequences of interest. The algorithm of Karlin and Altschul (Karlin and Altschul, Proc. Natl. Acad. Sci. USA 87:22264-2268, 1990) modified as in Karlin and Altschul, Proc. Natl. Acad. Sci. USA 90:5873-5877, 1993 is incorporated into the NBLAST and XBLAST programs of Altschul et al. (Altschul, et al., J. Mol. Biol. 215:403-410, 1990). To obtain gapped alignments for comparison purposes, Gapped BLAST is utilized as described in Altschul et al. (Altschul, et al. Nucleic Acids Res. 25: 3389-3402, 1997). When utilizing BLAST and Gapped BLAST programs, the default parameters of the respective programs may be used. A PAM250 or BLOSUM62 matrix may be used. Software for performing BLAST analyses is publicly available through the National Center for Biotechnology Information (NCBI). See the Web site having URL www.ncbi.nlm.nih.gov for these programs. In a specific embodiment, percent identity is calculated using BLAST2 with default parameters as provided by the NCBI.

“Isolated” or “partially purified” as used herein refers, in the case of a nucleic acid or polypeptide, to a nucleic acid or polypeptide separated from at least one other component (e.g., nucleic acid or polypeptide) that is present with the nucleic acid or polypeptide as found in its natural source and/or that would be present with the nucleic acid or polypeptide when expressed by a cell, or secreted in the case of secreted polypeptides. A chemically synthesized nucleic acid or polypeptide or one synthesized using in vitro transcription/translation is considered “isolated”. An “isolated cell” is a cell that has been removed from an organism in which it was originally found or is a descendant of such a cell. Optionally the cell has been cultured in vitro, e.g., in the presence of other cells. Optionally the cell is later introduced into a second organism or re-introduced into the organism from which it (or the cell from which it is descended) was isolated.

“Modulate” is used consistently with its use in the art, i.e., meaning to cause or facilitate a qualitative or quantitative change, alteration, or modification in a process, pathway, or phenomenon of interest. Without limitation, such change may be an increase, decrease, or change in relative strength or activity of different components or branches of the process, pathway, or phenomenon. A “modulator” is an agent that causes or facilitates a qualitative or quantitative change, alteration, or modification in a process, pathway, or phenomenon of interest.

The term “pluripotency factor” is used refer to an expression product of a pluripotency gene. If the pluripotency gene encodes a protein, the term “pluripotency factor” typically refers to the protein but may refer to the mRNA encoding the protein.

“Pluripotency gene”, as used herein, refers to a gene whose expression under normal conditions (e.g., in the absence of genetic engineering or other manipulation designed to alter gene expression) occurs in and is typically restricted to pluripotent stem cells, and is crucial for their functional identity as such. It will be appreciated that the polypeptide encoded by a pluripotency gene may be present as a maternal factor in the oocyte. The gene may be expressed by at least some cells of the embryo, e.g., throughout at least a portion of the preimplantation period and/or in germ cell precursors of the adult. The gene may be expressed in ES cells and/or in embryonic carcinoma cells. The pluripotency gene is typically substantially not expressed in somatic cell types that constitute the body of an adult animal under normal conditions (with the exception of germ cells or precursors thereof, or possibly in certain disease states such as cancer). For example, the pluripotency gene may be one whose average expression level (based on RNA or protein) in ES cells is at least 50-fold or 100-fold greater than its average level in those terminally differentiated cell types present in the body of an adult mammal. In some embodiments, the pluripotency gene is one that encodes multiple splice variants or isoforms of a protein, wherein one or more such variants or isoforms is expressed in at least some adult somatic cell types, while one or more other variants or isoforms is not substantially expressed in adult somatic cells under normal conditions. In some embodiments, expression of the pluripotency gene is essential to maintain the viability or pluripotent state of ES cells. Thus if the gene is knocked out or its expression is inhibited (i.e., its expression is eliminated or substantially reduced, e.g., such that the average steady state level of RNA transcript and/or protein encoded by the gene is decreased by at least 50%, 60%, 70%, 80%, 90%, 95%, or more), the ES cells are not formed, die or, in some embodiments, differentiate or cease to be pluripotent. In some embodiments the pluripotency gene is characterized in that its expression in an ES cell or iPS cell decreases (resulting in, e.g., a reduction in the average steady state level of RNA transcript and/or protein encoded by the gene by at least 50%, 60%, 70%, 80%, 90%, 95%, or more) when the cell differentiates into a terminally differentiated cell. Oct4 and Nanog are exemplary pluripotency genes.

“Reprogramming factor” refers to a gene, RNA, or protein that promotes or contributes to cell reprogramming, e.g., in vitro. Many useful reprogramming factors are transcription factors. In aspects of the invention relating to reprogramming factor(s), the invention provides embodiments in which the reprogramming factor(s) are of interest for reprogramming somatic cells to pluripotency in vitro. Examples of reprogramming factors of interest for reprogramming somatic cells to pluripotency in vitro are Oct4, Nanog, Sox2, Lin28, Klf4, c-Myc, and any gene/protein that can substitute for one or more of these in a method of reprogramming somatic cells in vitro. “Reprogramming to a pluripotent state in vitro”, “reprogramming to a pluripotency in vitro”, is used herein to refer to in vitro reprogramming methods that do not require and typically do not include nuclear or cytoplasmic transfer or cell fusion, e.g., with oocytes, embryos, germ cells, or pluripotent cells. Any embodiment or claim of the invention may specifically exclude compositions or methods relating to or involving nuclear or cytoplasmic transfer or cell fusion, e.g., with oocytes, embryos, germ cells, or pluripotent cells.

“Reprogramming protocol” refers to any treatment or combination of treatments that causes at least some cells to become reprogrammed. In some embodiments, “reprogramming protocol” can refer to a variation of a known reprogramming protocol, wherein a factor or other agent used in a known reprogramming protocol is omitted or modified. In some embodiments, “reprogramming protocol” can refer to a variation of a known reprogramming protocol, wherein a factor or agent known to be of use for reprogramming is used together with a different agent whose utility in reprogramming has not been established.

“RNA interference” is used herein consistently with its meaning in the art to refer to a phenomenon whereby double-stranded RNA (dsRNA) triggers the sequence-specific degradation or translational repression of a corresponding mRNA having complementarity to a strand of the dsRNA. It will be appreciated that the complementarity between the strand of the dsRNA and the mRNA need not be 100% but need only be sufficient to mediate inhibition of gene expression (also referred to as “silencing” or “knockdown”). For example, the degree of complementarity is such that the strand can either (i) guide cleavage of the mRNA in the RNA-induced silencing complex (RISC); or (ii) cause translational repression of the mRNA. In certain embodiments the double-stranded portion of the RNA is less than about 30 nucleotides in length, e.g., between 17 and 29 nucleotides in length. In mammalian cells, RNAi may be achieved by introducing an appropriate double-stranded nucleic acid into the cells or expressing a nucleic acid in cells that is then processed intracellularly to yield dsRNA therein. Nucleic acids capable of mediating RNAi are referred to herein as “RNAi agents”. Exemplary nucleic acids capable of mediating RNAi are a short hairpin RNA (shRNA), a short interfering RNA (siRNA), and a microRNA precursor. These terms are well known and are used herein consistently with their meaning in the art. siRNAs typically comprise two separate nucleic acid strands that are hybridized to each other to form a duplex. They can be synthesized in vitro, e.g., using standard nucleic acid synthesis techniques. They can comprise a wide variety of modified nucleosides, nucleoside analogs and can comprise chemically or biologically modified bases, modified backbones, etc. Any modification recognized in the art as being useful for RNAi can be used. Some modifications result in increased stability, cell uptake, potency, etc. In certain embodiments the siRNA comprises a duplex about 19 nucleotides in length and one or two 3′ overhangs of 1-5 nucleotides in length, which may be composed of deoxyribonucleotides. shRNA comprise a single nucleic acid strand that contains two complementary portions separated by a predominantly non-selfcomplementary region. The complementary portions hybridize to form a duplex structure and the non-selfcomplementary region forms a loop connecting the 3′ end of one strand of the duplex and the 5′ end of the other strand. shRNAs undergo intracellular processing to generate siRNAs.

“Selectable marker” refers to a gene, RNA, or protein that when expressed, confers upon cells a selectable phenotype, such as resistance to a cytotoxic or cytostatic agent (e.g., antibiotic resistance), nutritional prototrophy, or expression of a particular protein that can be used as a basis to distinguish cells that express the protein from cells that do not. Proteins whose expression can be readily detected such as a fluorescent or luminescent protein or an enzyme that acts on a substrate to produce a colored, fluorescent, or luminescent substance (“detectable markers”) constitute a subset of selectable markers. The presence of a selectable marker linked to expression control elements native to a gene that is normally expressed selectively or exclusively in pluripotent cells makes it possible to identify and select somatic cells that have been reprogrammed to a pluripotent state. A variety of selectable marker genes can be used, such as neomycin resistance gene (neo), puromycin resistance gene (puro), guanine phosphoribosyl transferase (gpt), dihydrofolate reductase (DHFR), adenosine deaminase (ada), puromycin-N-acetyltransferase (PAC), hygromycin resistance gene (hyg), multidrug resistance gene (mdr), thymidine kinase (TK), hypoxanthine-guanine phosphoribosyltransferase (HPRT), and hisD gene. Detectable markers include green fluorescent protein (GFP) blue, sapphire, yellow, red, orange, and cyan fluorescent proteins and variants of any of these. Luminescent proteins such as luciferase (e.g., firefly or Renilla luciferase) are also of use. As will be evident to one of skill in the art, the term “selectable marker” as used herein can refer to a gene or to an expression product of the gene, e.g., an encoded protein.

“Small molecule” refers to an organic compound having multiple carbon-carbon bonds and a molecular weight of less than 1500 daltons. Typically such compounds comprise one or more functional groups that mediate structural interactions with proteins, e.g., hydrogen bonding, and typically include at least an amine, carbonyl, hydroxyl or carboxyl group, and in some embodiments at least two of the functional chemical groups. The small molecule agents may comprise cyclic carbon or heterocyclic structures and/or aromatic or polyaromatic structures substituted with one or more chemical functional groups and/or heteroatoms.

“Somatic cell” refers to any cell other than a germ cell, a cell present in or obtained from a pre-implantation embryo, or a cell resulting from proliferation of such a cell in vitro. In some embodiments the somatic cell is a “non-embryonic somatic cell”, by which is meant a somatic cell that is not present in or obtained from an embryo and does not result from proliferation of such a cell in vitro. In some embodiments the somatic cell is an “adult somatic cell”, by which is meant a cell that is present in or obtained from an organism other than an embryo or a fetus or results from proliferation of such a cell in vitro.

The terms “treat”, “treating”, “treatment”, etc., as applied to an isolated cell, include subjecting the cell to any kind of process or condition or performing any kind of manipulation or procedure on the cell. As applied to a subject, the terms refer to providing medical or surgical attention, care, or management to an individual. The individual is usually ill (suffers from a disease or other condition warranting medical/surgical attention) or injured, or at increased risk of becoming ill relative to an average member of the population and in need of such attention, care, or management. “Individual” is used interchangeably with “subject” herein. In any of the embodiments of the invention, the “individual” may be a human, e.g., one who suffers or is at risk of a disease for which cell therapy is of use (“indicated”).

Overview

The present invention relates to compositions and methods for reprogramming mammalian cells. The ability to reprogram cell type provides, among other things, a means to generate immune-compatible cells for personalized regenerative medicine. Certain methods of the present invention facilitate generating autologous pluripotent cells. Certain methods of the present invention facilitate generating autologous differentiated cells of a desired cell type. The autologous cells are derived from somatic cells obtained from the individual. In general, autologous cells are less likely than non-autologous cells to be subject to immune rejection.

Reprogramming, as used herein, refers to a process that alters the differentiation state or identity of a cell. Cells are classified into different “types” based on various criteria such as morphological and functional characteristics and gene expression profile. “Cell state” encompasses the concept of “cell type” or “cell identity” but also refers to any one or more features or characteristics (or sets of features or characteristics) that characterize a cell (e.g., pluripotent state, differentiated state, post-mitotic state, etc.). In some embodiments, the invention provides methods for reprogramming somatic cells to a less differentiated state. The resulting cells are referred to herein as “reprogrammed somatic cells” (“RSC”). The reprogrammed cells are also referred to as “ES-like” or induced pluripotent stem (iPS) cells if they are pluripotent. In some embodiments, reprogramming entails complete reversion of the differentiation state of a somatic cell to a pluripotent state, in which the cell has the ability to differentiate into or give rise to cells derived from all three embryonic germ layers (endoderm, mesoderm and ectoderm) and typically has the potential to divide in vitro for a long period of time, e.g., greater than one year or more than 30 passages. In some embodiments, reprogramming entails partial reversion of the differentiation state of a differentiated somatic cell to a multipotent state, in which the cell is able to differentiate into some but not all of the cells derived from all three germ layers. In some embodiments, reprogramming entails differentiating a pluripotent cell (e.g., an iPS cell) or multipotent cell to a more differentiated cell of a desired cell type. In some embodiments, reprogramming entails converting a cell of a first differentiated cell type into a cell of a second differentiated cell type (also referred to as “trans-differentiation”), without apparently going through an intermediate stage of pluripotency. Unless otherwise indicated, the methods for reprogramming cells are performed in vitro, i.e., they are practiced using cells maintained in culture.

Screen for Regulators of Pluripotency

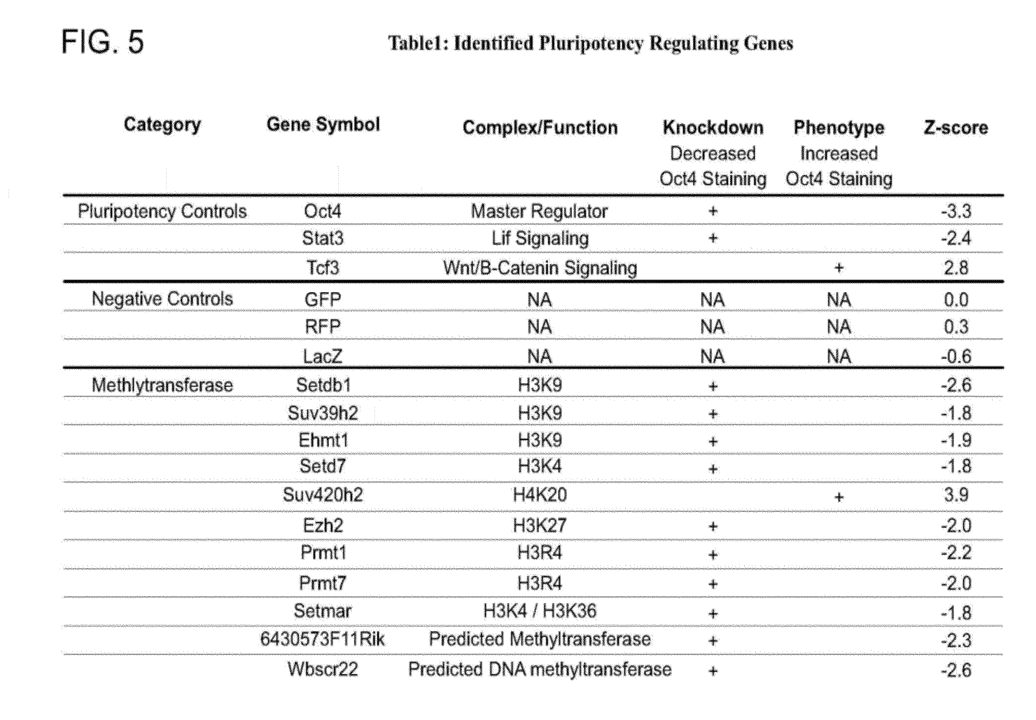

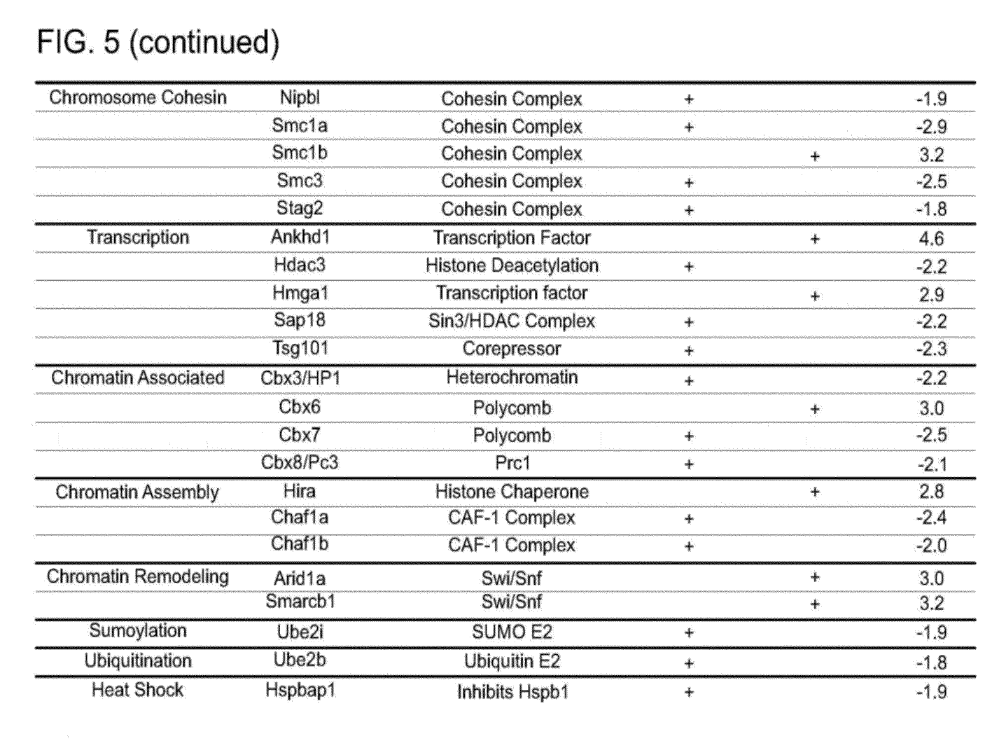

The regulation of gene expression in embryonic stem (ES) cells is a dynamic process that involves the expression of pluripotency genes and the silencing of developmental regulator genes to maintain the cell in an undifferentiated state. As ES cells differentiate, this transcriptional program must be altered to activate lineage specific genes. A set of key transcription factors and signaling pathways have been implicated in controlling these processes (see, e.g., Jaenisch, R., and Young, R. A. (2008) and references therein). However, a global view of the complete network of transcription factors and signaling components that regulate ES cell pluripotency and developmental potential does not exist. Applicants reasoned that identifying genes involved in regulating pluripotency in ES cells would provide insight into methods of modulating cell reprogramming. Applicants undertook a screen to identify genes involved in the regulation of pluripotency in ES cells. As described in Example 1, the inventive approach involved inhibiting gene expression in ES cells using shRNAs targeted against individual genes and assessing the effect on expression of the pluripotency gene Oct4. Applicants thereby identified genes whose inhibition either promoted differentiation (decreased Oct4 expression) or resulted in cells that are less primed to differentiate (increased Oct4 expression). Certain of the genes of particular interest are listed in Table 1 (FIG. 5) and/or Table 2 and discussed further below. Although Oct4 expression was used as an indicator of pluripotency, any of a number of different markers of pluripotency could be used. The marker may, but need not be, a pluripotency factor. In some embodiments, the marker is encoded by a gene whose expression is under control of a pluripotency factor. In some embodiments of the method, siRNA are used rather than shRNA. Libraries of shRNA or siRNA of use in the method are commercially available. Applicants' initial experiments were performed using shRNA designed to inhibit genes encoding proteins associated functionally and/or physically with chromatin, e.g., proteins associated with assembly, remodeling, modification, structure, etc., of one or more chromatin components—DNA, histone(s), or non-histone protein(s), but the inventive method may be employed using siRNA or shRNA designed to inhibit any gene of interest

Reprogramming and Methods of Enhancing Reprogramming

The Applicants reasoned that modulating activity of genes that regulate pluripotency in ES cells would modulate efficiency of in vitro reprogramming methods. As described in Examples 2 and 3, Applicants showed that inhibiting expression of certain genes that were identified in the inventive screen as genes whose inhibition promotes ES cell differentiation resulted in increased reprogramming efficiency, thereby confirming the utility of the inventive method. In particular, Applicants discovered that inhibiting expression of methyltransferases (e.g., histone methyltransferases) promotes differentiation of pluripotent cells. Applicants further discovered that inhibiting expression of certain of these histone methyltransferases increased the efficiency of reprogramming somatic cells to pluripotency. Applicants' results that establish an important role for histone methylation and the cellular machinery involved in histone methylation (e.g., histone methyltransferases, proteins that recruit histone methyltransferases to their target, etc.) in regulating pluripotency and, more generally, in regulating cell differentiation. Applicants' results establish that modulating histone methylation, e.g., by modulating activity of certain histone methyltransferases (HMTs), is of use to modulate reprogramming of somatic cells to pluripotency and/or to modulate reprogramming pluripotent cells to differentiated cells of a desired cell type.

Histones are a highly conserved family of proteins rich in lysine and arginine. Two copies of each of the four core histone proteins (H2A, H2B, H3, and H4) form an octameric structure that wraps 147 base pairs of eukaryotic DNA into a nucleosome. Histone proteins are extensively post-translationally modified at a number of residues. The role of such modifications in regulating chromatin structure and function and the proteins that accomplish such modifications are areas of active research (see, e.g., Smith, B C and Denu, J M; Biochim Biophys Acta. 2008 Jun. 14. [Epub ahead of print]). Certain lysine residues in histones can undergo methylation of their ε-amine groups. Histone-specific protein lysine methyltranseferases (HKMTs) belong to a novel 5-adenosyl methionine-dependent lysine methyltransferase family whose members share (in almost all cases) a conserved catalytic motif known as the SET domain. Methylation by different members of this family occurs at H1K26 (catalyzed by EZH2); H3K4 (catalyzed by the Set1, Set7/9, ASH1, SMYD3, and MLL enzymes); H3K9 (catalyzed by Suv39h1, Suv39h2, G9a, ESET (SetDB1), RIZ1, ASH1, and GLP/Eu-HMTase); H3K27 (catalyzed by EZH1, EZH2; G9a); H3K36 (catalyzed by NSD1 and HIF1); H3K79 (catalyzed by DOT1L); H4K20 (catalyzed by PR-Set7/Set8, Suv420h1 and Suv420h2, MLL), wherein the foregoing names refer to mammalian, e.g., human, HKMTs. It will be appreciated that the afore-mentioned lists are non-limiting and represent only a subset of the histone lysine methyltransferases. Certain arginine methyltransferase proteins (HRMTs) methylate particular arginine residues in histones. For example, PRMT1 is a histone methyltransferase that methylates Arg3 on histone H4. It will be appreciated that histone monomethylation, dimethylation, or trimethylation can occur, and different enzymes may catalyze one or more of these reactions and may associate in different protein complexes. Different methylation states of multiple histone lysines have distinct biological distributions in chromatin in at least some cell types and are associated with a variety of functional consequences (e.g., transcriptional activation, transcriptional silencing, heterochromatic silencing, DNA methylation, etc.), in ways that are not fully elucidated. HMTs are discussed in more detail in, e.g., Couture, J-F. and Trievel, R C, Curr Op. Struct. Biol., 2006; 16:753-760; Qian, C. and Zhou, M. M., Cell and Mol. Life. Sci., 2006; 63: 2755-2763; Gibbons, R., Hum. Mol. Genet., 2005; 14(1): R85-R92; Daniel, J A, et al., Cell Cycle, 4(7): 919-926). The mRNA and protein sequences of the afore-mentioned HMTs and others are known in the art, and those of skill in the art will readily be able to locate such sequences in publicly available databases.

The present invention establishes an important role for histone methylation and histone methyltransferase enzymes in regulating pluripotency and reprogramming. As described in Examples 1, 2, and 3, inhibiting histone methyltransferases, particularly H3K9 methyltransferases, promoted differentiation of ES cells while also promoting reprogramming of differentiated cells to a pluripotent state. Results in Example 1 showed that inhibiting histone methyltransferase activity in pluripotent cells promotes cell differentiation (and its accompanying loss of pluripotency), while results presented in Examples 2 and 3 indicate that inhibiting histone methyltransferase activity in differentiated cells promotes reprogramming to pluripotency, increasing the efficiency with which expression of reprogramming factors drives cells toward the pluripotent state. Applicants showed that an increased number of iPS cell colonies comprised of iPS cells developed when somatic cells genetically engineered to express Oct4, Sox2, and Klf4 were cultured in medium containing siRNA targeted to various H3K9 methyltransferases than when the cells were cultured in medium lacking such siRNA. Applicants further showed that these cells exhibited expression of the ES cell marker SSEA1. By all criteria tested, the cells appear to be identical to iPS cells generated by other means.

Without wishing to be bound by any theory, Applicants reasoned that, taken collectively, the results indicate that inhibiting histone methylation (e.g., H3K9 methylation) helps facilitate changes in cell state, e.g., makes cells more susceptible to undergoing a change in cell state in the presence of appropriate inducer(s) or other conditions favoring such a change, or on a stochastic basis if cells continually have a finite “baseline” probability of undergoing a change in cell state. Hence the effect of such inhibition may depend on the initial state of the cells and the conditions to which they are exposed. According to this interpretation, inhibiting histone methyltransferase activity in pluripotent cells would render them more likely to differentiate (consistent with loss of Oct4 staining in Example 1), while inhibiting histone methyltransferase activity in differentiated cells should render them more susceptible to reprogramming, as was shown to be the case in Examples 2 and 3. The effect of inhibiting histone methylation is likely to depend on context and presence of reprogramming factors or other agents that promote pluripotency or differentiation. Thus, Applicants results demonstrate that inhibiting histone methylation, e.g., by inhibiting histone methyltransferase activity, is a broadly useful approach to promoting reprogramming of cells to a desired state or cell type.

The invention provides a method of modulating the reprogramming of mammalian cells comprising: (a) modulating histone methylation in the cells; and (b) subjecting the cells to a reprogramming protocol so that at least some cells become reprogrammed to a desired cell state, wherein modulating histone methylation in the cells modulates reprogramming. The invention further provides a method of enhancing the reprogramming of mammalian cells comprising: (a) inhibiting histone methylation in the cells; and (b) subjecting the cells to a reprogramming protocol so that at least some cells become reprogrammed to a desired cell state, wherein inhibiting histone methylation in the cells enhances reprogramming. The invention further provides a method of enhancing the reprogramming of mammalian cells comprising: (a) inhibiting activity of an HMT in the cells; and (b) subjecting the cells to a reprogramming protocol so that at least some cells become reprogrammed to a desired cell state, wherein inhibiting activity of an HMT enhances reprogramming. The invention provides a method of enhancing the reprogramming of mammalian cells comprising: (a) contacting mammalian cells with an agent that inhibits histone methylation; and (b) subjecting the cells to a reprogramming treatment so that at least some cells become reprogrammed to a desired cell state, wherein the agent enhances such reprogramming. The invention provides a method of enhancing the reprogramming of mammalian cells comprising: (a) contacting mammalian cells with an agent that inhibits HMT activity; and (b) subjecting the cells to a reprogramming treatment so that at least some cells become reprogrammed to a desired cell state, wherein the agent enhances reprogramming. In some embodiments, inhibiting HMT activity comprises inhibiting HMT expression. In the afore-mentioned methods, the HMT may be an HKMT, e.g., an H3K9 MT.

The invention provides a method of modulating the reprogramming of mammalian cells comprising: (a) modulating activity of a gene listed in Table 2 or Table 3 in the cells; and (b) subjecting the cells to a reprogramming protocol so that at least some cells become reprogrammed to a desired cell state, wherein modulating activity of a gene listed in Table 2 or Table 3 modulates reprogramming. The invention further provides a method of enhancing the reprogramming of mammalian cells comprising: (a) inhibiting expression of a gene listed in Table 1 in the cells; and (b) subjecting the cells to a reprogramming protocol so that at least some cells become reprogrammed to a desired cell state, wherein inhibiting expression of a gene listed in Table 1 in the cells modulates reprogramming. The invention further provides a method of enhancing the reprogramming of mammalian cells comprising: (a) inhibiting expression of a gene listed in Table 2 in the cells; and (b) subjecting the cells to a reprogramming protocol so that at least some cells become reprogrammed to a desired cell state, wherein inhibiting expression of a gene listed in Table 2 in the cells enhances reprogramming. Some embodiments of the invention relate to modulating activity of a single gene listed in Table 1, 2, and/or 3. Other embodiments relate to modulating activity of multiple genes listed in Tables 1, 2, and/or 3.

Cells may be treated in any of a variety of ways to cause reprogramming according to the methods of the present invention. The treatment can comprise contacting the cells with one or more agent(s) that contribute to reprogramming (“reprogramming agent”). Such contacting may be performed by maintaining the cell in culture medium comprising the agent(s). In some embodiments the somatic cells are genetically engineered. The somatic cell may be genetically engineered to express one or more reprogramming factor(s) as described herein and known in the art. Either prior to or during at least part of the reprogramming treatment, cells are contacted with an agent that modulates, e.g., inhibits, histone methylation. In accordance with the inventive methods, such contacting modulates, e.g., enhances, reprogramming. For example, such agent may increase reprogramming efficiency and/or speed or allows generation of reprogrammed cells under conditions in which detectable generation of reprogrammed cells would not otherwise occur. In some embodiments, “increase the efficiency of reprogramming” encompasses causing an increase in the percentage of cells that undergo reprogramming to a desired cell state or cell type (e.g., to iPS cells) when a population of cells is subjected to a reprogramming treatment, typically resulting in a greater number of individual colonies of reprogrammed cells after a given time period, than would otherwise be the case (“colony enrichment”). For example, the number of colonies may be increased by a factor (“enrichment factor”) of at least 2, e.g., between 2 and 50, e.g., about 2, 4, 8, 16, etc. In some embodiments, the inventive methods decrease the amount of time required to obtain at least some reprogrammed cells or decrease the amount of time required to obtain a given number of colonies of reprogrammed cells from a given number of somatic cells. For example, such time may be decreased by at least 1, 2, 3, 4, or 5 days, or more. In some embodiments of the invention, wherein it is desired to reprogram somatic cells to iPS cells, somatic cells are treated (e.g., genetically engineered) so that they express one or more reprogramming factors selected from: Sox2, Klf family members (e.g., Klf2, Klf4), Oct4, Nanog, Lin28, and c-Myc at levels greater than would be the case in the absence of such treatment (i.e., they “overexpress” the factor(s). In some embodiments of the invention the cells are treated so that they overexpress Sox2, Klf4, Oct4, and c-Myc. In some embodiments of the invention the cells are treated so that they overexpress Sox2, Klf4, and Oct4 (or any subset thereof) but are not genetically engineered to overexpress c-Myc. In some embodiments of the invention the cells are treated so that they overexpress Oct4, Nanog, Sox2, and Lin28. Suitable methods of engineering such expression include infecting cells with viruses (e.g., retrovirus, lentivirus) or transfecting the cells with viral vectors (e.g., retroviral, lentiviral) that contain the sequences of the factors operably linked to suitable expression control elements to drive expression in the cells following infection or transfection and, optionally integration into the genome as known in the art. The invention provides the recognition that inhibiting histone methylation, e.g., H3K9 methylation, enhances reprogramming of somatic cells that have not been genetically modified to increase their expression of an oncogene such as c-Myc. The invention thus provides ways to substitute for engineered expression of c-Myc in any method of reprogramming somatic cells that would otherwise involve engineering cells to express c-Myc.

Without wishing to be bound by theory, it is possible that reducing histone methylation, e.g., histone lysine methylation, e.g., H3K9 methylation, facilitates the activity of agents, factors, or conditions that induce or promote alterations in cell state. For example, reducing histone methylation lower the threshold level or activity required for such agents, factors, or conditions to effectively impose other chromatin modifications or alterations in gene transcription that establish a different cell state. Accordingly, the inventive method would be of use to facilitate converting differentiated cells of a first cell type into differentiated cells of a second cell type, e.g., by expressing the appropriate reprogramming factors therein or by contacting the cells with agent(s) that act on the appropriate pathways.

One aspect of the invention relates to transient inhibition of histone methylation. Without wishing to be bound by theory, it is suggested that inhibiting histone methylation for a limited time period may facilitate allowing cells in a first state to enter a state that is permissive for establishing a second, different, cell state. However, in order to effectively establish the second cell state, it may be important to allow histone methylation to proceed. Accordingly, the invention encompasses transient inhibition of histone methylation under conditions suitable for reprogramming, and then relieving inhibition to allow establishment of a stable second cell state.

In some embodiments of the inventive methods, a single HMT is modulated. In some embodiments, the HMT is inhibited. “Inhibition” may be achieved by inhibiting activity or expression. For purposes of convenience, “inhibiting HMT activity” will be used herein to refer to inhibiting activity of an HMT protein or inhibiting HMT expression (e.g., by causing mRNA degradation, inhibiting mRNA translation, etc.). In some embodiments, the HMT is an HKMT. In some embodiments the HKMT is an H3K9 MT. In some embodiments the H3K9 MT is a Suv39h MT. In some embodiments the H3K9 MT is a Suv39h1. In some embodiments the H3K9 MT is Suv39h2. In some embodiments the H3K9 MT is SetDB1. In some embodiments the H3K9 MT is Ehmt1. In some embodiments, at least two HKMTs (e.g., 2, 3, 4, etc.) are inhibited. For example, both Suv39h1 and Suv39h2 are inhibited in some embodiments. In certain embodiments of the invention histone monomethylation (e.g., H3K9 monomethylation) is inhibited. In certain embodiments histone dimethylation (e.g., H3K9 dimethylation) is inhibited. In certain embodiments histone trimethylation (e.g., H3K9 trimethylation) is inhibited. In some embodiments, the HMT is not G9a. In some embodiments, the HMT is not an H4K20 MT. In some embodiments, the HTM is not Suv420h2. In some embodiments, expression of an H4K20 is enhanced. In some embodiments, expression of a Suv420h2 is enhanced. In some embodiments, the HMT is an HRMT. In some embodiments, the HMRT is an H3R4 methyltransferase. In some embodiments, the HRMT is PRMT1. In some embodiments the HRMT is PRMT7.

Inhibiting histone methylation may be accomplished in a variety of ways and may employ a variety of different agents. In some embodiments histone methyltransferase activity is inhibited using RNAi. shRNA may be expressed intracellularly, or cells may be cultured in medium containing siRNA. In some embodiments an inhibitor of use in the present invention is an RNAi agent. One of skill in the art will be able to identify an appropriate RNAi agent to inhibit expression of a gene of interest. In some embodiments of the invention, the RNAi agent inhibits expression sufficiently to reduce the average steady state level of the RNA transcribed from the gene (e.g., mRNA) or its encoded protein by, e.g., by at least 50%, 60%, 70%, 80%, 90%, 95%, or more). The RNAi agent may contain a sequence between 15-29 nucleotides long, e.g., 17-23 nucleotides long, e.g., 19-21 nucleotides long, that is 100% complementary to the mRNA or contains up to 1, 2, 3, 4, or 5 nucleotides, or up to about 10-30% nucleotides, that do not participate in Watson-Crick base pairs when aligned with the mRNA to achieve the maximum number of complementary base pairs. The RNAi agent may contain a duplex between 17-29 nucleotides long in which all nucleotides participate in Watson-Crick base pairs or in which up to about 10-30% of the nucleotides do not participate in a Watson-Crick base pair. One of skill in the art will be aware of which sequence characteristics are often associated with superior siRNA functionality and will be aware of algorithms and rules by which such siRNAs can be designed (see, e.g., Jagla, B., et al, RNA, 11(6):864-72, 2005). The methods of the invention can employ siRNAs having such characteristics. In some embodiments the sequence of either or both strands of the RNAi agent is/are chosen to avoid silencing non-target genes, e.g., the strand(s) may have less than 70%, 80%, or 90% complementarity to any mRNA other than the target mRNA. In some embodiments multiple different sequences are used. RNAi agents capable of silencing mammalian genes are commercially available (e.g., from suppliers such as Qiagen, Dharmacon, Ambion/ABI, Sigma-Aldrich, etc.). If multiple isoforms of a gene of interest exist, one can design siRNAs or shRNAs targeted against a region present in all of the isoforms expressed in a given cell of interest.

Methods for silencing genes by transfecting cells with siRNA or constructs encoding shRNA are known in the art. To express an RNAi agent in somatic cells, a nucleic acid construct comprising a sequence that encodes the RNAi agent, operably linked to suitable expression control elements, e.g., a promoter, can be introduced into the cells as known in the art. For purposes of the present invention a nucleic acid construct that comprises a sequence that encodes an RNA or polypeptide of interest, the sequence being operably linked to expression control elements such as a promoter that direct transcription in a cell of interest, is referred to as an “expression cassette”. The promoter can be an RNA polymerase I, II, or III promoter functional in somatic mammalian cells. In certain embodiments expression of the RNAi agent is conditional. In some embodiments expression is regulated by placing the sequence that encodes the RNAi agent under control of a regulatable (e.g., inducible or repressible) promoter. Example 2 discloses sequences for certain siRNAs that were shown to be effective in inhibiting expression of their target HMT. In some embodiments of the invention, an siRNA disclosed in Example 2 (or shRNA based on the same sequences) is used. In some embodiments, an siRNA having an antisense strand disclosed in Example 2 is used. One of skill in the art will be able to identify siRNA sequences that target corresponding regions of human orthologs. In some embodiments an siRNA or shRNA that targets a single HMT is used. The antisense strand may be complementary to a region that is not found in other HMT mRNA sequences. In some embodiments a combination of two or more siRNAs or shRNAs targeted to a single HMT is used. In some embodiments an siRNA or shRNA designed to inhibit multiple HMTs is used. For example, the siRNA or shRNA may target a region that is conserved among multiple HMTs.

In some embodiments, histone methylation is decreased by increasing histone demethylase activity in the cell. Histone demethylating enzymes are known in the art (see, e.g., Cloos, P A, et al., Genes Dev. 2008 May 1; 22(9):1115-40). In some embodiments, histone demethylase activity is increased by introducing a histone demethylase enzyme or a nucleic acid construct containing a gene encoding a histone demethylase enzyme into cells. In some embodiments, expression of the HMT or histone demethylase is normally at least partly repressed by an endogenous microRNA (miRNA). Expression of such proteins can be enhanced by inhibiting the miRNA. A miRNA can be inhibited by introducing an antisense oligonucleotide that hybridizes to the miRNA into a cell.

In some embodiments cells are treated to enhance uptake of an agent that acts intracellularly. For example, the cell membrane may be partially permeabilized. In some embodiments a polypeptide agent is modified to comprise an amino acid sequence that enhances cellular uptake of molecules by cells (also referred to as a “protein transduction domain”). Such uptake-enhancing amino acid sequences are found, e.g., in HIV-1 TAT protein, the herpes simplex virus 1 (HSV-1) DNA-binding protein VP22, the Drosophila Antennapedia (Antp) transcription factor, etc. Artificial sequences are also of use. See, e.g., Fischer et al, Bioconjugate Chem., Vol. 12, No. 6, 2001 and U.S. Pat. No. 6,835,810.

In some embodiments of the invention, cells are contacted with an HMT modulator for a time period of at least 1 days while in other embodiments the period of time is at least 3, 5, 10, 15, or 20 days. In some embodiments, cells are contacted for at least 1 and no more than 3, 5, 10, 15, or 20 days.

In certain embodiments of the invention the HMT inhibitor is a protein, small molecule, or aptamer. In some embodiments, the agent (e.g., protein, small molecule, or aptamer) binds to and inhibits a HMT or binds to and inhibits a protein whose activity is needed for HMT activity. Small molecule inhibitors of various HMTs are may be used in various embodiments of the invention. In some embodiments the HMT inhibitor is an analog of S-adenosyl methionine or competes with S-adenosyl methionine. An example of such a compound is 5″-deoxy-5″-(methylthio)adenosine. Certain HMKT inhibitors are described in the following: Greiner, D, et al., Nat Chem. Biol. 2005 August; 1(3):143-5, which describes the fungal metabolite chaetocin as the first inhibitor of a lysine-specific histone methyltransferase. Chaetocin is specific for the methyltransferase SU(VAR)3-9 both in vitro and in vivo; Kubicek, S., et al., Mol. Cell. 2007 Feb. 9; 25(3):473-81, describing a screen for specific inhibitors against histone lysine methyltransferases (HMTases) using recombinant G9a as the target enzyme and identification of 7 compounds of which one, BIX-01294 (a diazepine-quinazoline-amine derivative), does not compete with the cofactor S-adenosyl-methionine, and selectively impairs the G9a HMTase and the generation of H3K9me2 in vitro. In some embodiments, however, the molecule is not BIX-01294. In some embodiments the compound is not a compound in the same structural class as BIX-02194. WO2008001391 (PCT/IN2007/000258) discloses, among other things, various compounds isolated from pomegranates, and derivatives, that inhibit certain HMTs.

The invention encompasses testing histone methylation inhibitors, e.g., libraries of small molecules known or suspected to inhibit histone methylation (e.g., histone methyltransferase inhibitors), to identify those that are effective in enhancing reprogramming and/or have superior ability to enhance reprogramming, e.g., relative to other compounds tested. In some embodiments, at least 10, at least 20, at least 50, at least 100, or at least 1,000 small molecules, e.g., structurally related molecules, at least some of which are known or believed to inhibit histone methylation, are tested.

In some embodiments the concentration of the modulator (e.g., inhibitor) added to the medium is between 10 and 10,000 ng/ml, e.g., between 100 and 5,000 ng/ml, e.g., between 1,000 and 2,500 ng/ml or between 2,500 and 5,000 ng/ml, or between 5,000 and 10,000 ng/ml.

Methods of the invention may include treating the cells with multiple agents either concurrently (i.e., during time periods that overlap at least in part) or sequentially and/or repeating the steps of treating the cells with an agent. The agent used in the repeating treatment may be the same as, or different from, the one used during the first treatment.

The cells may be contacted with a reprogramming agent for varying periods of time. In some embodiments the cells are contacted with the agent for a period of time between 1 hour and 60 days, e.g., between 10 and 30 days, e.g., for about 15-20 days. Reprogramming agents may be added each time the cell culture medium is replaced. The reprogramming agent(s) may be removed prior to performing a selection to enrich for pluripotent cells or assessing the cells for pluripotency characteristics.

Somatic cells of use in the invention may be primary cells (non-immortalized cells), such as those freshly isolated from an animal, or may be derived from a cell line capable or prolonged proliferation in culture (e.g., for longer than 3 months) or indefinite proliferation (immortalized cells). Adult somatic cells may be obtained from individuals, e.g., human subjects, and cultured according to standard cell culture protocols available to those of ordinary skill in the art. The cells may be maintained in cell culture following their isolation from a subject. In certain embodiments the cells are passaged once or more following their isolation from the individual (e.g., between 2-5, 5-10, 10-20, 20-50, 50-100 times, or more) prior to their use in a method of the invention. They may be frozen and subsequently thawed prior to use. In some embodiments the cells will have been passaged no more than 1, 2, 5, 10, 20, or 50 times following their isolation from the individual prior to their use in a method of the invention. In some embodiments, methods of the invention utilize cells of a cell line, e.g., a population of largely or substantially identical cells that have typically been derived from a single ancestor cell or from a defined and/or substantially identical population of ancestor cells or from a tissue sample obtained from a particular individual. The cell line may have been or may be capable of being maintained in culture for an extended period (e.g., months, years, for an unlimited period of time). It may have undergone a spontaneous or induced process of transformation conferring an unlimited culture lifespan on the cells. Cell lines include all those cell lines recognized in the art as such. It will be appreciated that cells acquire mutations and possibly epigenetic changes over time such that at least some properties of individual cells of a cell line may differ with respect to each other.

Somatic cells of use in the present invention are typically mammalian cells, such as, for example, human cells, non-human primate cells, or mouse cells. They may be obtained by well-known methods from various organs, e.g., skin, lung, pancreas, liver, stomach, intestine, heart, reproductive organs, bladder, kidney, urethra and other urinary organs, etc., generally from any organ or tissue containing live somatic cells. Mammalian somatic cells useful in various embodiments of the present invention may be fibroblasts, adult stem cells, sertoli cells, granulosa cells, neurons, pancreatic islet cells, epidermal cells, epithelial cells, endothelial cells, hepatocytes, hair follicle cells, keratinocytes, hematopoietic cells, melanocytes, chondrocytes, lymphocytes (B and T lymphocytes), macrophages, monocytes, mononuclear cells, cardiac muscle cells, skeletal muscle cells, etc., generally any nucleated living somatic cells. In some embodiments, the somatic cell is a terminally differentiated cell, i.e., the cell is fully differentiated and does not (under normal conditions in the body) give rise to more specialized cells. In some embodiments the somatic cell is a terminally differentiated cell that does not divide under normal conditions in the body, i.e., the cell cannot self-renew. In some embodiments, the somatic cell is a precursor cell, i.e., the cell is not fully differentiated and is capable of giving rise to cells that are more fully differentiated. In some embodiments, cells that can be obtained relatively convenient procedure from a human subject are used (e.g., fibroblasts, keratinocytes, circulating white blood cells).

Genetically homogeneous ‘secondary’ somatic cells that carry reprogramming factors as defined doxycycline (dox)-inducible transgenes are of use in certain embodiments of the invention (See, e.g., Wernig, et al., A novel drug-inducible transgenic system for direct reprogramming of multiple somatic cell types. Nature Biotechnology, 2008 August; 26(8):916-24. Epub 2008 Jul. 1.). These cells may be produced by infecting fibroblasts with dox-inducible lentiviruses carrying genes encoding the reprogramming factors, reprogramming by dox addition, selecting induced pluripotent stem cells and using such cells to produce chimeric mice. Somatic cells derived from these chimeras (“secondary somatic cells”, e.g., secondary mouse embryonic fibroblasts) reprogram upon dox exposure without the need for viral infection with efficiencies 25- to 50-fold greater than those observed using direct infection and drug selection for pluripotency marker reactivation. These “secondary iPS cells” are genetically homogeneous with respect to the viral integration sites. In some embodiments, one can differentiate the initial iPS cells in vitro by withdrawing inducer, isolate individual cells, and establish a genetically homogeneous cell line therefrom. In some embodiments, secondary somatic cells generated without use of c-Myc virus are used.

One can generate somatic cells that have a subset of the reprogramming factors necessary to achieve reprogramming under control of a first inducible promoter and the remaining factor(s) (e.g., any individual factor) under control of a second inducible promoter. One can then generate iPS cells by inducing expression from both promoters. One can then withdraw the inducers, allowing the cells to differentiate, thereby generating “secondary” somatic cells. One can subsequently apply one of the inducers, thereby inducing expression of only a subset of the reprogramming factors, and test candidate agents to identify ones that substitute for expression of the remaining factor(s). For example, one could generate a genetically homogeneous population of somatic cells that express any 1, 2, or 3 reprogramming factors and screen to identify agents that substitute for the other factor(s). The invention provides compositions comprising such “secondary” cells and a histone methylation inhibitor. In certain embodiments the composition further comprises a candidate reprogramming agent.

In some embodiments of the invention the somatic cells contain a nucleic acid sequence encoding a selectable marker, operably linked to a promoter of an endogenous gene of interest, wherein expression of the gene of interest occurs specifically or selectively in cells of a desired type. Expression of the selectable marker is of use to identify cells that have been reprogrammed to a desired type and to identify reprogramming agents. For example, if the desired cell type is a pluripotent cell, the gene may be an endogenous pluripotency gene, e.g., Oct4 or Nanog. The sequence encoding the marker may be integrated into the genome at the endogenous locus. The selectable marker may be, e.g., a readily detectable protein such as a fluorescent protein, e.g., GFP or a derivative thereof. Expression of the marker is indicative of reprogramming and can thus be used to identify or select reprogrammed cells, quantify reprogramming efficiency, and/or to identify, characterize, or use agents that enhance reprogramming and/or are being tested for their ability to enhance reprogramming.

In some embodiments the methods are practiced using somatic cells that are not genetically engineered for purposes of identifying or selecting reprogrammed cells. The resulting reprogrammed somatic cells do not contain exogenous genetic material that has been introduced into said cells (or ancestors of said cells) by the hand of man, e.g., for purposes of identifying or selecting reprogrammed cells. In some embodiments the somatic cells and reprogrammed somatic cells derived therefrom do contain exogenous genetic material in their genome, but such genetic material is introduced for purposes of correcting a genetic defect in such cells or enabling such cells to synthesize a desired protein for therapeutic purposes and is not used to identify or select reprogrammed cells.

Reprogramming Protocols

To reprogram somatic cells to pluripotency, the cells may be treated to cause them to express or contain one or more reprogramming factor or pluripotency factor at levels greater than would be the case in the absence of such treatment. For example, somatic cells may be genetically engineered to express one or more genes encoding one or more such factor(s) and/or may be treated with agent(s) that increase expression of one or more endogenous genes encoding such factors and/or stabilize such factor(s). The agent could be, for example, a small molecule, a nucleic acid, a polypeptide, etc. In some embodiments, pluripotency factors are introduced into somatic cells, e.g., by microinjection or by contacting the cells with the factors under conditions in which the factors are taken up by the cells. In some embodiments the factors are modified to incorporate a protein transduction domain. In some embodiments the cells are permeabilized or otherwise treated to increase their uptake of the factors. Exemplary factors are discussed below.

The transcription factor Oct4 (also called Pou5fl, Oct-3, Oct3/4) is an example of a pluripotency factor. Oct4 has been shown to be required for establishing and maintaining the undifferentiated phenotype of ES cells and plays a major role in determining early events in embryogenesis and cellular differentiation (Nichols et al., 1998, Cell 95:379-391; Niwa et al., 2000, Nature Genet. 24:372-376). Oct4 expression is down-regulated as stem cells differentiate into more specialized cells. Nanog is another example of a pluripotency factor. Nanog is a homeobox-containing transcription factor with an essential function in maintaining the pluripotent cells of the inner cell mass and in the derivation of ES cells from these. Furthermore, overexpression of Nanog is capable of maintaining the pluripotency and self-renewing characteristics of ESCs under what normally would be differentiation-inducing culture conditions. (See Chambers et al., 2003, Cell 113: 643-655; Mitsui et al., Cell. 2003, 113(5):631-42). Sox2, another pluripotency factor, is an HMG domain-containing transcription factor known to be essential for normal pluripotent cell development and maintenance (Avilion, A., et al., Genes Dev. 17, 126-140, 2003). Klf4 is a Krüppel-type zinc finger transcription factor initially identified as a Klf family member expressed in the gut (Shields, J. M, et al., J. Biol. Chem. 271:20009-20017, 1996). Overexpression of Klf4 in mouse ES cells was found to prevent differentiation in embryoid bodies formed in suspension culture, suggesting that Klf4 contributes to ES self renewal (Li, Y., et al., Blood 105:635-637, 2005). Sox2 is a member of the family of SOX (sex determining region Y-box) transcription factors and is important for maintaining ES cell self-renewal. c-Myc is a transcription factor that plays a myriad of roles in normal development and physiology as well as being an oncogene whose dysregulated expression or mutation is implicated in various types of cancer (reviewed in Pelengaris S, Khan M., Arch Biochem Biophys. 416(2):129-36, 2003; Cole M D, Nikiforov M A, Curr Top Microbiol Immunol., 302:33-50, 2006). In some embodiments such factors are selected from the group consisting of: Oct4, Sox2, Klf4, and combinations thereof. In some embodiments a different, functionally overlapping Klf family member such as Klf2 is substituted for Klf4. In some embodiments the factors include at least Oct4. In some embodiments the factors include at least Oct4 and a Klf family member, e.g., Klf2. Lin28 is a developmentally regulated RNA binding protein. In some embodiments somatic cells are treated so that they express or contain one or more reprogramming factors selected from the group consisting of: Oct4, Sox2, Klf4, Nanog, Lin28, and combinations thereof. CCAAT/enhancer-binding-protein-alpha (C/EBPalpha) is another protein that promotes reprogramming at least in certain cell types, e.g., lymphoid cells such as B-lineage cells, is considered a reprogramming factor for such cell types.

In one embodiment, the exogenously introduced gene may be expressed from a chromosomal locus other than the chromosomal locus of an endogenous gene whose function is associated with pluripotency. Such a chromosomal locus may be a locus with open chromatin structure, and contain gene(s) whose expression is not required in somatic cells, e.g., the chromosomal locus contains gene(s) whose disruption will not cause cells to die. Exemplary chromosomal loci include, for example, the mouse ROSA 26 locus and type II collagen (Col2a1) locus (See Zambrowicz et al., 1997).

Methods for expressing genes in cells are known in the art. Generally, a sequence encoding a polypeptide or functional RNA such as an RNAi agent is operably linked to appropriate regulatory sequences (e.g., promoters, enhancers and/or other expression control elements). Exemplary regulatory sequences are described in Goeddel; Gene Expression Technology: Methods in Enzymology, Academic Press, San Diego, Calif. (1990).

The gene may be expressed from an inducible or repressible regulatory sequence such that its expression can be regulated. Exemplary inducible promoters include, for example, promoters that respond to heavy metals (CRC Boca Raton, Fla. (1991), 167-220; Brinster et al. Nature (1982), 296, 39-42), to thermal shocks, to hormones (Lee et al. P.N.A.S. USA (1988), 85, 1204-1208; (1981), 294, 228-232; Klock et al. Nature (1987), 329, 734-736; Israel and Kaufman, Nucleic Acids Res. (1989), 17, 2589-2604), promoters that respond to chemical agents, such as glucose, lactose, galactose or antibiotics. A tetracycline-inducible promoter is an example of an inducible promoter that responds to an antibiotic (tetracycline or an analog thereof). See Gossen, M. and Bujard, H., Annu Rev Genet. Vol. 36: 153-173 2002 and references therein. Tetracycline analog includes any compound that displays structural similarity with tetracycline and is capable of activating a tetracycline-inducible promoter. Exemplary tetracycline analogs include, for example, doxycycline, chlorotetracycline and anhydrotetracycline.

In some embodiments of the invention expression of an introduced gene, e.g., a gene encoding a reprogramming factor or RNAi agent is transient. Transient expression can be achieved by transient transfection or by expression from a regulatable promoter. In some embodiments expression can be regulated by, or is dependent on, expression of a site-specific recombinase. Recombinase systems include the Cre-Lox and Flp-Frt systems, among others (Gossen, M. and Bujard, H., 2002). In some embodiments a recombinase is used to turn on expression by removing a stopper sequence that would otherwise separate the coding sequence from expression control sequences. In some embodiments a recombinase is used to excise at least a portion of a gene after reprogramming has been induced. In some embodiments the recombinase is expressed transiently, e.g., it becomes undetectable after about 1-2 days, 2-7 days, 1-2 weeks, etc. In some embodiments the recombinase is introduced from external sources.

It is contemplated that protein reprogramming factors (e.g., Oct4, Sox2, Klf4, etc.) may be introduced into cells, thereby avoiding introducing exogenous genetic material. Such proteins may be modified to include a protein transduction domain. Such uptake-enhancing amino acid sequences are found, e.g., in HIV-1 TAT protein, the herpes simplex virus 1 (HSV-1) DNA-binding protein VP22, the Drosophila Antennapedia (Antp) transcription factor, etc. Artificial sequences are also of use. See, e.g., Fischer et al, Bioconjugate Chem., Vol. 12, No. 6, 2001 and U.S. Pat. No. 6,835,810.

It is contemplated that a variety of additional agents may be of use to enhance reprogramming. Such agents may be used in combination with an HMT modulator, e.g., HMT inhibitor. Exemplary agents are agents that inhibit histone deacetylation, e.g., histone deacetylase (HDAC) inhibitors and agents that inhibit DNA methylation, e.g., DNA methyltransferase inhibitors. Major classes of HDAC inhibitors include (a) Small chain fatty acids (e.g., valproic acid); (b) hydroxamate small molecule inhibitors (e.g., SAHA and PXD101); (c) Non-hydroxamate small molecule inhibitors, e.g., MS-275; and (d) Cyclic peptides: e.g., depsipeptide (see, e.g., Carey N and La Thangue N B, Curr Opin Pharmacol.; 6(4):369-75, 2006). Examples of histone deacetylase inhibitors are Trichostatin A: [R-(E,E)]-7-[4-(Dimethylamino)phenyl]-N-hydroxy-4,6-dimethyl-7-oxo-2,4-heptadienamide, which inhibits histone deacetylase at nanomolar concentrations; (Yoshida, M., et al., Bioessays 17, 423-430, 1995; Minucci, S., et al., Proc. Natl. Acad. Sci. USA 94, 11295-11300, 1997; Brehm, A., et al., 1998; Medina, V., et al., Cancer Res. 57, 3697-3707, 1997; Kim, M. S., et al., Cancer Res. 63, 7291-7300, 2003); and Apicidin: Cyclo[(2S)-2-amino-8-oxodecanoyl-1-methoxy-L-tryptophyl-L-isoleucyl-(2R)-2-piperidinexcarbonyl](Kwon, S. H., et al. J. Biol. Chem. 18, 2073, 2002; Han, J. W., et al. Cancer Res. 60, 6068, 2000; Colletti, S. L., et al. Bioorg. Med. Chem. 11, 107, 2001; Kim, J. S., et al. Biochem. Biophys. Res. Commun. 281, 866, 2001).

A variety of DNA methylation inhibitors are known in the art and are of use in certain embodiments of the invention. See, e.g., Lyko, F. and Brown, R., JNCI Journal of the National Cancer Institute, 97(20):1498-1506, 2005. Inhibitors of DNA methylation include nucleoside DNA methyltransferase inhibitors such as decitabine (2′-deoxy-5-azacytidine), 5-azadeoxycytidine, and zebularine, non-nucleoside inhibitors such as the polyphenol (−)-epigallocatechin-3-gallate (EGCG) and the small molecule RG108 (2-(1,3-dioxo-1,3-dihydro-2H-isoindol-2-yl)-3-(1H-indol-3-yl)propanoic acid), compounds described in WO2005085196 and phthalamides, succinimides and related compounds as described in WO2007007054. Three additional classes of compounds are: (1) 4-Aminobenzoic acid derivatives, such as the antiarrhythmic drug procainamide and the local anesthetic procaine; (2) the psammaplins, which also inhibit histone deacetylase (Pina, I. C., J Org. Chem., 68(10):3866-73, 2003); and (3) oligonucleotides, including siRNAs, shRNAs, and specific antisense oligonucleotides, such as MG98. DNA methylation inhibitors may act by a variety of different mechanisms. In some embodiments of the invention combinations of histone methylation inhibitor and a DNA methylation inhibitor are used. In some embodiments agents that incorporate into DNA (or whose metabolic products incorporate into DNA) are not used. DNA methyltransferase (DNMT1, 3a, and/or 3b) and/or one or more HDAC family members can alternatively or additionally be inhibited using RNAi agents. The invention provides a composition comprising a cell to be reprogrammed, a histone methylation inhibitor, and a DNA methylation inhibitor. The invention further provides a composition comprising a cell to be reprogrammed, a histone methylation inhibitor, and a histone deacetylase inhibitor.

While the present disclosure has focused on reprogramming somatic cells to pluripotency, the inventive methods may be applied to reprogram differentiated somatic cells from a first cell type to a second cell type. For example, it is contemplated that modulating genes and processes identified herein, e.g., inhibiting histone methylation, will enhance reprogramming protocols that involve expressing particular combinations of transcription factors in cells to convert them into cells of a different type. Such reprogramming protocols involving modulation of genes identified herein, e.g., inhibition of HMT activity, are an aspect of the invention.

In the methods of the present invention somatic cells may, in general, be cultured under standard conditions of temperature, pH, and other environmental conditions, e.g., as adherent cells in tissue culture plates at 37° C. in an atmosphere containing 5-10% CO₂. The cells and/or the cell culture medium are appropriately modified to achieve reprogramming as described herein. The cell culture medium contains nutrients that are sufficient to maintain viability and, typically, support proliferation of at least some cell types. The medium may contain any of the following in an appropriate combination: salt(s), buffer(s), amino acids, glucose or other sugar(s), antibiotics, serum or serum replacement, and other components such as peptide growth factors, etc. Cell culture media ordinarily used for particular cell types are known to those skilled in the art. Some non-limiting examples are provided herein.

In some embodiments, somatic cells are reprogrammed to iPS cells. In some embodiments, such cells are cultured in medium suitable for culturing ES cells while undergoing reprogramming. Exemplary serum-containing ES medium is made with 80% DMEM (typically KO DMEM), 20% defined fetal bovine serum (FBS) not heat inactivated, 1% non-essential amino acids, 1 mM L-glutamine, and 0.1 mM 3-mercaptoethanol. The medium is filtered and stored at 4° C., e.g., for 2 weeks or less. Serum-free ES medium may be prepared with 80% KO DMEM, 20% serum replacement, 1% non-essential amino acids, 1 mM L-glutamine, and 0.1 mM (3-mercaptoethanol and a serum replacement such as Invitrogen Cat. No. 10828-028. The medium is filtered and stored at 4° C. Before combining with the cells used for conditioning, human bFGF can be added to a final concentration of 4 ng/mL. StemPro® hESC SFM (Invitrogen Cat. No. A1000701), a fully defined, serum- and feeder-free medium (SFM) specially formulated for the growth and expansion of human embryonic stem cells, is of use. In some embodiments, iPS cells are reprogrammed to one or more differentiated cell types. The iPS cells may be cultured initially in medium suitable for maintaining ES cells and may be transferred to medium suitable for the desired cell type.

In certain embodiments the cells are cultured on or in the presence of a material that mimics one or more features of the extracellular matrix or comprises one or more extracellular matrix or basement membrane components. In some embodiments Matrigel™ is used. Other materials include proteins or mixtures thereof such as gelatin, collagen, fibronectin, etc. In certain embodiments of the invention the cells are cultured in the presence of a feeder layer of cells. Such cells may, for example, be of murine or human origin. They may be irradiated, chemically inactivated by treatment with a chemical inactivator such as mitomycin c, or otherwise treated to inhibit their proliferation if desired. In other embodiments the somatic cells are cultured without feeder cells.

Assessing Reprogramming Efficiency

Reprogrammed somatic cells may be assessed for one or more characteristics of a desired cell state or cell type. For example, cells may be assessed for pluripotency characteristic(s). The presence of pluripotency characteristic(s) indicates that the somatic cells have been reprogrammed to a pluripotent state. The term “pluripotency characteristics”, as used herein, refers to characteristics associated with and indicative of pluripotency, including, for example, the ability to differentiate into cells derived from all three embryonic germ layers all types and a gene expression pattern distinct for a pluripotent cell, including expression of pluripotency factors and expression of other ES cell markers.

To assess potentially reprogrammed somatic cells for pluripotency characteristics, one may analyze such cells for particular growth characteristics and ES cell-like morphology. Cells may be injected subcutaneously into immunocompromised SCID mice to determine whether they induce teratomas (a standard assay for ES cells). ES-like cells can be differentiated into embryoid bodies (another ES specific feature). Moreover, ES-like cells can be differentiated in vitro by adding certain growth factors known to drive differentiation into specific cell types. Self-renewing capacity, marked by induction of telomerase activity, is another plutipotency characteristic that can be monitored. One may carry out functional assays of the reprogrammed somatic cells by introducing them into blastocysts and determining whether the cells are capable of giving rise to all cell types. See Hogan et al., 2003. If the reprogrammed cells are capable of forming a few cell types of the body, they are multipotent; if the reprogrammed cells are capable of forming all cell types of the body including germ cells, they are pluripotent.

One may also examine the expression of an individual pluripotency factor. Additionally or alternately, one may assess expression of other ES cell markers such as stage-specific embryonic 1 5 antigens-1, -3, and -4 (SSEA-1, SSEA-3, SSEA-4), which are glycoproteins specifically expressed in early embryonic development and are markers for ES cells (Salter and Knowles, 1978, Proc. Natl. Acad. Sci. USA 75:5565-5569; Kannagi et al., 1983, EMBO J. 2:2355-2361). Elevated expression of the enzyme alkaline phosphatase (AP) is another marker associated with undifferentiated embryonic stem cells (Wobus et al., 1 984, Exp. Cell 152:212-219; Pease et al., 1990, Dev. Biol. 141:322-352). Additional ES cell markers are described in Ginis, I., et al., Dev. Biol., 269: 369-380, 2004 and in The International Stem Cell Initiative, Adewumi O, et al., Nat. Biotechnol., 25(7):803-16, 2007 and references therein. For example, TRA-1-60, TRA-1-81, GCTM2 and GCT343, and the protein antigens CD9, Thy1 (CD90), class 1 HLA, NANOG, TDGF1, DNMT3B, GABRB3 and GDF3, REX-1, TERT, UTF-1, TRF-1, TRF-2, connexin43, connexin45, Foxd3, FGFR-4, ABCG-2, and Glut-1 are of use.

One may perform expression profiling of the reprogrammed somatic cells to assess their pluripotency characteristics. Pluripotent cells, such as embryonic stem cells, and multipotent cells, such as adult stem cells, are known to have a distinct pattern of global gene expression. See, for example, Ramalho-Santos et al., Science 298: 597-600, 2002; Ivanova et al., Science 298: 601-604, 2002; Boyer, L A, et al. Nature 441, 349, 2006, and Bernstein, B E, et al., Cell 125 (2), 315, 2006. One may assess DNA methylation, gene expression, and/or epigenetic state of cellular DNA, and/or developmental potential of the cells, e.g., as described in Wernig, M., et al., Nature, 448:318-24, 2007. Cells that are able to form teratomas containing cells having characteristics of endoderm, mesoderm, and ectoderm when injected into SCID mice and/or possess ability to participate (following injection into murine blastocysts) in formation of chimeras that survive to term are considered pluripotent. Another method of use to assess pluripotency is determining whether the cells have reactivated a silent X chromosome.

Similar methods may be used to assess efficiency of reprogramming cells to a desired cell type or lineage. Expression of markers that are selectively or specifically expressed in such cells may be assessed. For example, markers expressed selectively or specifically by neural, hematopoietic, myogenic, or other cell lineages and differentiated cell types are known, and their expression can be assessed. In some embodiments of the invention the expression level of 2-5, 5-10, 10-25, 25-50, 50-100, 100-250, 250-500, 500-1000, or more RNAs (e.g., mRNAs) or proteins is increased by reprogramming the cell according to the methods of the invention. Functional or morphological characteristics of the cells can be assessed to evaluate the efficiency of reprogramming.

Certain methods of the invention include a step of identifying or selecting cells that express a marker that is expressed by multipotent or pluripotent cells or by cells of a desired cell type or lineage. Standard cell separation methods, e.g., flow cytometry, affinity separation, etc. may be used. Alternately or additionally, one could select cells that do not express markers characteristic of the cells from which the potentially reprogrammed cells were derived. Other methods of separating cells may utilize differences in average cell size or density that may exist between pluripotent cells and somatic cells. For example, cells can be filtered through materials having pores that will allow only certain cells to pass through.

In some embodiments the somatic cells contain a nucleic acid comprising regulatory sequences of a gene encoding a pluripotency factor operably linked to a selectable or detectable marker (e.g., GFP or neo). The nucleic acid sequence encoding the marker may be integrated at the endogenous locus of the gene encoding the pluripotency factor (e.g., Oct4, Nanog) or the construct may comprise regulatory sequences operably linked to the marker. Expression of the marker may be used to select, identify, and/or quantify reprogrammed cells.

Any of the methods of the invention that relate to generating a reprogrammed somatic cell may include a step of obtaining a somatic cell or obtaining a population of somatic cells from an individual in need of cell therapy. Reprogrammed somatic cells are generated, selected, or identified from among the obtained cells or cells descended from the obtained cells. Optionally the cell(s) are expanded in culture prior to generating, selecting, or identifying reprogrammed somatic cell(s) genetically matched to the donor.

In some embodiments colonies are subcloned and/or passaged once or more in order to obtain a population of cells enriched for desired cells, e.g., iPS cells. The enriched population may contain at least 95%, 96%, 97%, 98%, 99% or more, e.g., 100% cells of a desired type. The invention provides cell lines of somatic cells that have been stably and heritably reprogrammed to an ES-like state.

In some embodiments, the methods employ morphological criteria to identify reprogrammed cells from among a population of cells that are not reprogrammed to a desired type. In some embodiments, the methods employ morphological criteria to identify somatic cells that have been reprogrammed to an ES-like state from among a population of cells that are not reprogrammed or are only partly reprogrammed to an ES-like state. “Morphological criteria” is used in a broad sense to refer to any visually detectable feature or characteristic of the cells or colonies. Morphological criteria include, e.g., the shape of the colonies, the sharpness of colony boundaries, the density, small size, and rounded shape of the cells relative to non-reprogrammed cells, etc. For example, dense colonies composed of small, rounded cells, and having sharp colony boundaries are characteristic of ES and iPS cells. The invention encompasses identifying and, optionally, isolating colonies (or cells from colonies) wherein the colonies display one or more characteristics of a desired cell type. The reprogrammed somatic cells may be identified as colonies growing in a first cell culture dish (which term refers to any vessel, plate, dish, receptacle, container, etc., in which living cells can be maintained in vitro) and the colonies, or portions thereof, transferred to a second cell culture dish, thereby isolating reprogrammed cells. The cells may then be further expanded.

Methods of Screening for a Reprogramming Agent

The present invention also provides methods for identifying an agent that, alone or in combination with one or more other agents, reprograms somatic cells to a less differentiated state. The invention further provides agents identified according to the methods. In one embodiment, the methods comprise contacting somatic cells with a histone methylation inhibitor and a candidate agent and determining whether the presence of the candidate agent results in enhanced reprogramming relative to that which would occur if cells had not been contacted with the candidate agent. In some embodiments the histone methylation inhibitor and candidate agent are present together in the cell culture medium while in other embodiments the histone methylation inhibitor and the candidate agent are not present together (e.g., the cells are exposed to the agents sequentially). The cells may be maintained in culture for, e.g., at least 3 days, at least 5 days, up to 10 days, up to 15 days, up to 30 days, etc., during which time they are contacted with the histone methylation inhibitor and the candidate agent for all or part of the time. In some embodiments the agent is identified as a reprogramming agent if there are at least 2, 5, or 10 times as many reprogrammed cells or colonies comprising predominantly reprogrammed cells after said time period than if the cells have not been contacted with the candidate agent.

A candidate agent can be any molecule or supramolecular complex, e.g. a polypeptide, peptide (which herein refers to a polypeptide containing 60 amino acids or less), small organic or inorganic molecule (i.e., molecules having a molecular weight less than 1,500 Da, 1000 Da, or 500 Da in various embodiments), polysaccharide, polynucleotide, etc. which is to be tested for ability to reprogram cells In some embodiments, candidate agents are organic molecules, e.g., small organic molecules, comprising functional groups that mediate structural interactions with proteins, e.g., hydrogen bonding, and typically include at least an amine, carbonyl, hydroxyl or carboxyl group, and in some embodiments at least two of the functional chemical groups. The candidate agents may comprise cyclic carbon or heterocyclic structures and/or aromatic or polyaromatic structures substituted with one or more chemical functional groups and/or heteroatoms.

Candidate agents may be obtained from a wide variety of sources. In some embodiments, candidate agents are synthetic compounds. Numerous techniques are available for the random and directed synthesis of a wide variety of organic compounds and biomolecules. In some embodiments, the candidate modulators are provided as mixtures of natural compounds in the form of bacterial, fungal, plant and animal extracts, fermentation broths, conditioned media, etc., that are available or readily produced. In some embodiments, a library of compounds is screened. A library is typically a collection of compounds that can be presented or displayed such that the compounds can be identified in a screening assay. In some embodiments compounds in the library are housed in individual wells (e.g., of microtiter plates), vessels, tubes, etc., to facilitate convenient transfer to individual wells or vessels for contacting cells, performing cell-free assays, etc. The library may be composed of molecules having common structural features which differ in the number or type of group attached to the main structure or may be completely random. Libraries include but are not limited to, for example, phage display libraries, peptide libraries, polysome libraries, aptamer libraries, synthetic small molecule libraries, natural compound libraries, etc. Small molecules include organic molecules often having multiple carbon-carbon bonds. The libraries can comprise cyclic carbon or heterocyclic structure and/or aromatic or polyaromatic structures substituted with one or more functional groups. In some embodiments the small molecule has between 5 and 50 carbon atoms, e.g., between 7 and 30 carbons. In some embodiments the compounds are macrocyclic. Libraries of interest also include peptide or peptoid libraries, randomized oligonucleotide libraries, and the like. Small molecule combinatorial libraries may also be generated. A combinatorial library of small organic compounds may comprise a collection of closely related analogs that differ from each other in one or more points of diversity and are synthesized by organic techniques using multi-step processes. Representative libraries that could be screened are available from ChemBridge Corporation, 16981 Via Tazon, San Diego, Calif. 92127 (e.g., DIVERSet™) AnalytiCon USA Inc., P.O. Box 5926, Kingwood, Tex. 77325; 3-Dimensional Pharmaceuticals, Inc., 665 Stockton Drive, Suite 104, Exton, Pa. 19341-1151; Tripos, Inc., 1699 Hanley Rd., St. Louis, Mo., 63144-2913, etc. For example, libraries based on quinic acid and shikimic acid, hydroxyproline, santonine, dianhydro-D-glucitol, hydroxypipecolinic acid, andrographolide, piperazine-2-carboxylic acid based library, cytosine, etc., are commercially available. For descriptions of additional libraries, see, for example, Tan, et al., Am. Chem. Soc. 120, 8565-8566, 1998; Floyd C D, Leblanc C, Whittaker M, Prog Med Chem 36:91-168, 1999.

In some embodiments the candidate agents are cDNAs from a cDNA expression library prepared from cells, e.g., pluripotent cells. Such cells may be embryonic stem cells, oocytes, blastomeres, teratocarcinomas, embryonic germ cells, inner cell mass cells, etc.

The candidate reprogramming agent to be tested is typically one that is not present in standard culture medium, or if present is present in lower amounts than when used in the present invention.

A useful reprogramming treatment need not be capable of reprogramming all types of somatic cells and may reprogram only a fraction of somatic cells of a given cell type. A candidate agent that results in a population that is enriched for reprogrammed cells by a factor of 2, 5, 10, 50, 100 or more (i.e., the fraction of reprogrammed cells in the population, or the number of colonies of reprogrammed cells, is 2, 5, 10, 50, or 100 times more than would be present had the cells not been subjected to the reprogramming treatment population of cells treated in the same way but without being contacted with the candidate agent) is of use.

In some embodiments of the invention the inventive screening method is used to identify an agent or combination of agents that substitutes for Klf4 in reprogramming cells to an ES-like state. The method may be practiced using somatic cells engineered to express Sox2 and Oct4 and contacted with a histone methylation inhibitor and a candidate agent. In some embodiments, the method is used to identify an agent that substitutes for Sox2 in reprogramming cells to an ES-like state. The method may be practiced using somatic cells engineered to express Klf4 and Oct4 and contacted with a histone methylation inhibitor and a candidate agent. In some embodiments, the method is used to identify an agent that substitutes for Oct4 in reprogramming cells to an ES-like state. The method may be practiced using somatic cells engineered to express Sox2 and Klf 4 and contacted with a histone methylation inhibitor and a candidate agent. It is contemplated that genetically engineered expression of reprogramming factors is replaced by treating somatic cells with a combination of small molecules and/or polypeptides or other agents that do not modify the sequence of the genome. In some embodiments the methods are practiced using human cells. In some embodiments the methods are practiced using mouse cells. In some embodiments the methods are practiced using non-human primate cells. Compositions comprising cells described above and the above-mentioned combinations of agent(s) are aspects of the invention.

The methods and compositions of the present invention relating to histone methylation inhibitors may be applied to or used in combination with various other methods and compositions useful for cell reprogramming and/or for identifying reprogramming agents for use in somatic cell reprogramming. Such combined methods and compositions are aspects of the invention. For example, some embodiments of the invention employ cell types (e.g., neural stem cells or progenitor cells) that naturally express one or more reprogramming factors at levels higher than such factor(s) are expressed in many other cell types (see, e.g., Eminli, et al., Reprogramming of Neural Progenitor Cells into iPS Cells in the Absence of Exogenous Sox2 Expression. Stem Cells. 2008 Jul. 17., epub ahead of print).

The methods and compositions may be used together with methods and compositions disclosed in PCT/US2008/004516, which is incorporated herein by reference:

Methods for Gene Identification

The invention provides methods for identifying a gene whose expression inhibits generation of reprogrammed cells. One method comprises: (i) inhibiting histone methylation in somatic cells; (ii) reducing expression of a candidate gene by RNAi; (iii) determining whether reducing expression of the candidate gene results in increased efficiency of reprogramming and, if so, identifying the candidate gene as one whose expression inhibits reprogramming of somatic cells. Optionally the somatic cells are engineered to express at least one gene selected from: Oct4, Sox2, Nanog, Lin28, and Klf4 and combinations thereof (e.g., Oct4 and Sox2; Oct4 and Klf4). The identified gene is a target for inhibition in order to enhance cellular reprogramming. Agents that inhibit the gene (either RNAi agents or other agents such as small molecules) are of use to reprogram somatic cells.

Reprogrammed Somatic Cells and Uses Thereof

The present invention provides reprogrammed somatic cells (RSCs) produced by the methods of the invention. In some embodiments the RSCs are iPS cells. These cells have numerous applications in medicine, agriculture, and other areas of interest. The invention provides methods for the treatment or prevention of a condition in a mammal. In one embodiment, the methods involve obtaining somatic cells from the individual, reprogramming the somatic cells so obtained by methods of the present invention (e.g., in the presence of a histone methylation inhibitor) to obtain RSCs, e.g., iPS cells or cells of a desired cell type different to that of the harvested cells. In the case of iPS cells, in certain embodiments of the invention they are then cultured under conditions suitable for their development into cells of a desired cell type. The cells of the desired cell type are introduced into the individual to treat the condition. In an alternative embodiment, the methods start with obtaining somatic cells from the individual, reprogramming the somatic cells so obtained by methods of the present invention. The RPCs are then cultured under conditions suitable for development of the RPCs into a desired organ, which is harvested and introduced into the individual to treat the condition. The condition may be any condition in which cell or organ function is abnormal and/or reduced below normal levels. Thus the invention encompasses obtaining somatic cells from an individual in need of cell therapy, reprogramming the cells by a process that comprises inhibiting histone methylation in the cells, optionally differentiating reprogrammed somatic cells them to generate cells of one or more desired cell types, and introducing the cells into the individual. An individual in need of cell therapy may suffer from any condition, wherein the condition or one or more symptoms of the condition can be alleviated by administering cells to the donor and/or in which the progression of the condition can be slowed by administering cells to the individual. The method may include a step of identifying or selecting reprogrammed somatic cells and separating them from cells that are not reprogrammed.

The RSCs in certain embodiments of the present invention are ES-like cells, also referred to as iPS cells, and thus may be induced to differentiate to obtain the desired cell types according to known methods to differentiate ES cells. For example, the iPS cells may be induced to differentiate into hematopoietic stem cells, muscle cells, cardiac muscle cells, liver cells, pancreatic cells, cartilage cells, epithelial cells, urinary tract cells, nervous system cells (e.g., neurons) etc., by culturing such cells in differentiation medium and under conditions which provide for cell differentiation. Medium and methods which result in the differentiation of embryonic stem cells obtained using traditional methods are known in the art, as are suitable culturing conditions. Such methods and culture conditions may be applied to the iPS cells obtained according to the present invention. See, e.g., Trounson, A., The production and directed differentiation of human embryonic stem cells, Endocr Rev. 27(2):208-19, 2006 and references therein, all of which are incorporated by reference, for some examples. See also Yao, S., et al, Long-term self-renewal and directed differentiation of human embryonic stem cells in chemically defined conditions, Proc Natl Acad Sci USA, 103(18): 6907-6912, 2006 and references therein, all of which are incorporated by reference.

Thus, using known methods and culture medium, one skilled in the art may culture reprogrammed pluripotent cells to obtain desired differentiated cell types, e.g., neural cells, muscle cells, hematopoietic cells, etc. The subject cells may be used to obtain any desired differentiated cell type. Such differentiated human cells afford a multitude of therapeutic opportunities. For example, human hematopoietic stem cells derived from cells reprogrammed according to the present invention may be used in medical treatments requiring bone marrow transplantation. Such procedures are used to treat many diseases, e.g., late stage cancers and malignancies such as leukemia. Such cells are also of use to treat anemia, diseases that compromise the immune system such as AIDS, etc. The methods of the present invention can also be used to treat, prevent, or stabilize a neurological disease such as Alzheimer's disease, Parkinson's disease, Huntington's disease, or ALS, lysosomal storage diseases, multiple sclerosis, or a spinal cord injury. For example, somatic cells may be obtained from the individual in need of treatment, and reprogrammed to gain pluripotency, and cultured to derive neurectoderm cells that may be used to replace or assist the normal function of diseased or damaged tissue.

Reprogrammed cells that produce a growth factor or hormone such as insulin, etc., may be administered to a mammal for the treatment or prevention of endocrine disorders. Reprogrammed epithelial cells may be administered to repair damage to the lining of a body cavity or organ, such as a lung, gut, exocrine gland, or urogenital tract. It is also contemplated that reprogrammed cells may be administered to a mammal to treat damage or deficiency of cells in an organ such as the bladder, brain, esophagus, fallopian tube, heart, intestines, gallbladder, kidney, liver, lung, ovaries, pancreas, prostate, spinal cord, spleen, stomach, testes, thymus, thyroid, trachea, ureter, urethra, or uterus.

RSCs may be combined with a matrix to form a tissue or organ in vitro or in vivo that may be used to repair or replace a tissue or organ in a recipient mammal (such methods being encompassed by the term “cell therapy”). For example, RSCs may be cultured in vitro in the presence of a matrix to produce a tissue or organ of the urogenital, cardiovascular, or musculoskeletal system. Alternatively, a mixture of the cells and a matrix may be administered to a mammal for the formation of the desired tissue in vivo. The RSCs produced according to the invention may be used to produce genetically engineered or transgenic differentiated cells, e.g., by introducing a desired gene or genes, or removing all or part of an endogenous gene or genes of RSCs produced according to the invention, and allowing such cells to differentiate into the desired cell type. One method for achieving such modification is by homologous recombination, which technique can be used to insert, delete or modify a gene or genes at a specific site or sites in the genome.

This methodology can be used to replace defective genes or to introduce genes which result in the expression of therapeutically beneficial proteins such as growth factors, hormones, lymphokines, cytokines, enzymes, etc. For example, the gene encoding brain derived growth factor may be introduced into human embryonic or stem-like cells, the cells differentiated into neural cells and the cells transplanted into a Parkinson's patient to retard the loss of neural cells during such disease. Using known methods to introduced desired genes/mutations into ES cells, RSCs may be genetically engineered, and the resulting engineered cells differentiated into desired cell types, e.g., hematopoietic cells, neural cells, pancreatic cells, cartilage cells, etc. Genes which may be introduced into the RSCs include, for example, epidermal growth factor, basic fibroblast growth factor, glial derived neurotrophic growth factor, insulin-like growth factor (I and II), neurotrophin3, neurotrophin-4/5, ciliary neurotrophic factor, AFT-1, cytokine genes (interleukins, interferons, colony stimulating factors, tumor necrosis factors (alpha and beta), etc.), genes encoding therapeutic enzymes, collagen, human serum albumin, etc.

Negative selection systems known in the art can be used for eliminating therapeutic cells from a patient if desired. For example, cells transfected with the thymidine kinase (TK) gene will lead to the production of reprogrammed cells containing the TK gene that also express the TK gene. Such cells may be selectively eliminated at any time from a patient upon gancyclovir administration. Such a negative selection system is described in U.S. Pat. No. 5,698,446. In other embodiments the cells are engineered to contain a gene that encodes a toxic product whose expression is under control of an inducible promoter. Administration of the inducer causes production of the toxic product, leading to death of the cells. Thus any of the somatic cells of the invention may comprise a suicide gene, optionally contained in an expression cassette, which may be integrated into the genome. The suicide gene is one whose expression would be lethal to cells. Examples include genes encoding diphtheria toxin, cholera toxin, ricin, etc. The suicide gene may be under control of expression control elements that do not direct expression under normal circumstances in the absence of a specific inducing agent or stimulus. However, expression can be induced under appropriate conditions, e.g., (i) by administering an appropriate inducing agent to a cell or organism or (ii) if a particular gene (e.g., an oncogene, a gene involved in the cell division cycle, or a gene indicative of dedifferentiation or loss of differentiation) is expressed in the cells, or (iii) if expression of a gene such as a cell cycle control gene or a gene indicative of differentiation is lost. See, e.g., U.S. Pat. No. 6,761,884. In some embodiments the gene is only expressed following a recombination event mediated by a site-specific recombinase. Such an event may bring the coding sequence into operable association with expression control elements such as a promoter. Expression of the suicide gene may be induced if it is desired to eliminate cells (or their progeny) from the body of a subject after the cells (or their ancestors) have been administered to a subject. For example, if a reprogrammed somatic cell gives rise to a tumor, the tumor can be eliminated by inducing expression of the suicide gene. In some embodiments tumor formation is inhibited because the cells are automatically eliminated upon dedifferentiation or loss of proper cell cycle control.

Examples of diseases, disorders, or conditions that may be treated or prevented include neurological, endocrine, structural, skeletal, vascular, urinary, digestive, integumentary, blood, immune, auto-immune, inflammatory, endocrine, kidney, bladder, cardiovascular, cancer, circulatory, digestive, hematopoietic, and muscular diseases, disorders, and conditions. In addition, reprogrammed cells may be used for reconstructive applications, such as for repairing or replacing tissues or organs. In some embodiments, it may be advantageous to include growth factors and proteins or other agents that promote angiogenesis. Alternatively, the formation of tissues can be effected totally in vitro, with appropriate culture media and conditions, growth factors, and biodegradable polymer matrices.

The present invention contemplates all modes of administration, including intramuscular, intravenous, intraarticular, intralesional, subcutaneous, or any other route sufficient to provide a dose adequate to prevent or treat a disease. The RSCs may be administered to the mammal in a single dose or multiple doses. When multiple doses are administered, the doses may be separated from one another by, for example, one week, one month, one year, or ten years. One or more growth factors, hormones, interleukins, cytokines, or other cells may also be administered before, during, or after administration of the cells to further bias them towards a particular cell type.

The RSCs obtained using methods of the present invention may be used as an in vitro model of differentiation, e.g., for the study of genes which are involved in the regulation of early development. Differentiated cell tissues and organs generated using the reprogrammed cells may be used to study effects of drugs and/or identify potentially useful pharmaceutical agents.

Further Applications of Somatic Cell Reprogramming Methods and Reprogrammed Cells

The reprogramming methods disclosed herein may be used to generate RSCs, e.g., iPS cells, for a variety of animal species. The RSCs generated can be useful to produce desired animals. Animals include, for example, avians and mammals as well as any animal that is an endangered species. Exemplary birds include domesticated birds (e.g., chickens, ducks, geese, turkeys). Exemplary mammals include murine, caprine, ovine, bovine, porcine, canine, feline and non-human primate. Of these, preferred members include domesticated animals, including, for examples, cattle, pigs, horses, cows, rabbits, guinea pigs, sheep, and goats.

### EXEMPLIFICATION

The invention now being generally described, it will be more readily understood by reference to the following example, which are included merely for purposes of illustration of certain aspects and embodiments of the present invention, and are not intended to limit the invention.

### Example 1

### Screen to Identify Pluripotency Regulators

This Example describes an unbiased approach to identify transcriptional factors and signaling components involved in the regulation of pluripotency in ES cells. A short hairpin RNA (shRNA) library was used to perform a screen for factors that are involved in regulating pluripotency of mES cells. The lentiviral short hairpin RNA (shRNA) library targets 16,009 mouse genes, of which 200, 1316 and 1800 have been annotated as a chromatin factor, signaling component or transcription factor, respectively. On average 4-5 hairpins have been generated for each gene to provide redundancy and to address potential off-target effects. The library is described in Moffat, J., et al., Cell, 124(6):1283-98, 2006.

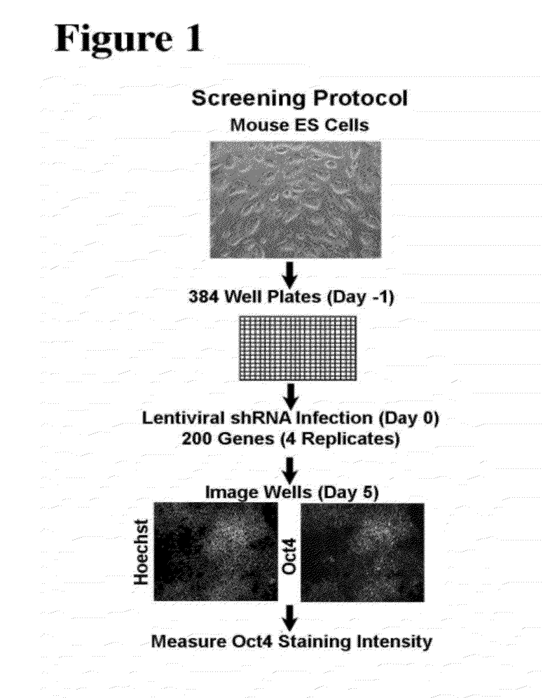

The initial screen was done with the chromatin factor set. FIG. 1 shows a schematic overview of the screen. Mouse embryonic stem cells were seeded in a 384 well plate and each well was infected with an individual shRNA. One day post infection, cells were treated with puromycin to select for the stable integration of the shRNA lentivirus. Five days post infection the cells were crosslinked and stained with Hoechst dye and for Oct4, a marker of pluripotency.

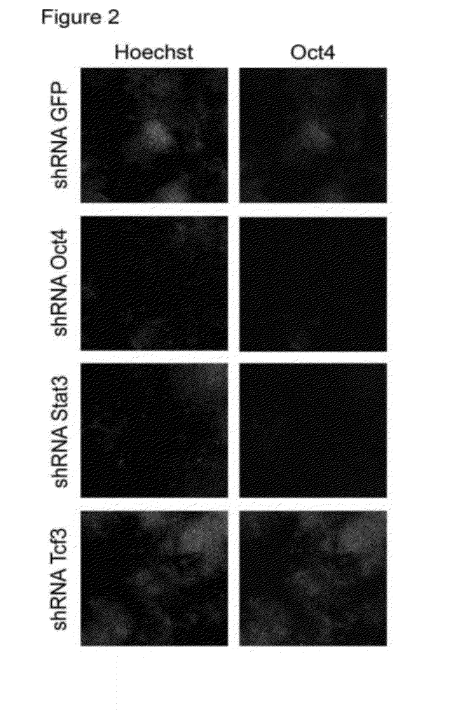

The plates were imaged using an ArrayScan (Cellomics) microscope, and the images were analyzed with the Cellomics software. The Cellomics software identified individual cells based on the Hoechst staining and then measured the average Oct4 staining intensity in each identified cell. [It will be appreciated that in some cases the software identifies small groups of cells that are too close together to be individually resolved.]] An average Oct4 staining intensity for the cells in the well was calculated. Hits were scored based on a significant increase (factors that prime mES cells for differentiation) or decrease (factors involved in maintaining pluripotency) in Oct4 staining intensity relative to infections with negative control virus (shRNAs targeting GFP, LacZ, and RFP). Each plate contained these negative control viruses. A decrease in Oct4 staining intensity served to indicate shRNA that induced the mES cells to differentiate. Likewise an increase in Oct4 intensity served to indicate that the shRNA has resulted in cells that are less primed to differentiate. Lentiviral shRNAs targeting Oct4 and Stat3 were included on each plate as positive controls since a reduced expression of either is known to cause mouse ES (mES) cells to differentiate and result in a decrease in Oct4 staining intensity. A lentiviral shRNA targeting Tcf3 was also included as a positive control since decreased expression of Tcf3 results in mES cells that are less prone to differentiation and an increase in Oct4 expression. FIG. 2 illustrates the positive and negative controls, demonstrating that the approach is capable of identifying shRNAs that either (i) inhibit differentiation (and thus help maintain pluripotency) or (ii) promote differentiation.

“Hits” were determined by measuring the average Oct4 staining intensity of all identified cells in a well. An individual Z-Score for that well was calculated from the negative controls (shRNAs targeting LacZ, RFP and GFP) on each plate. The Z-score represents the average of the four replicates.

Table 2 shows a partial list of knockdowns that induce differentiation (loss of Oct4 staining). An arbitrarily selected Z-score cutoff of −1.74 was used. The absolute value of the Z-score reflects the magnitude of the effect. Therefore, knockdowns with a more negative Z-score resulted in greater reduction in Oct4 staining. Some genes in the list appear more than once because on average there are 4-5 different shRNAs targeting a single gene in the library. Multiple hairpin hits may increase the likelihood that result reflects the effect of knocking down the target gene and serves as a means of validation. The CLONEID is used to distinguish between different hairpins targeting the same gene. Oct4_Spike In and Stat3_ Spike In refer to the positive control virus that was added to wells on every plate.

TABLE 2
Partial list of knockdowns that induce differentiation

| SYMBOL | CLONEID | Z-Score |
|---|---|---|
| Oct4 | Oct4_Spike In | −3.33 |
| Smc1a | TRCN0000109033 | −2.88 |
| Setdb1 | TRCN0000092975 | −2.62 |
| Smc1a | TRCN0000109034 | −2.56 |
| Wbscr22 | TRCN0000097425 | −2.56 |
| Cbx7 | TRCN0000096730 | −2.51 |
| Smc3 | TRCN0000109009 | −2.45 |
| Chaf1a | TRCN0000109035 | −2.41 |
| Stat3 | Stat3_Spike In | −2.37 |
| Wbscr22 | TRCN0000097428 | −2.36 |
| Tsg101 | TRCN0000054606 | −2.33 |
| 6430573F11Rik | TRCN0000125895 | −2.29 |
| Smc1a | TRCN0000109030 | −2.28 |
| Prmt1 | TRCN0000018492 | −2.20 |
| Sap18 | TRCN0000039377 | −2.19 |
| Smc3 | TRCN0000109007 | −2.18 |
| Hdac3 | TRCN0000039392 | −2.17 |
| Cbx3 | TRCN0000071038 | −2.17 |
| Cbx8 | TRCN0000093072 | −2.15 |
| Prmt7 | TRCN0000097476 | −2.02 |
| Ezh2 | TRCN0000039040 | −1.99 |
| Chaf1b | TRCN0000092872 | −1.99 |
| Hspbap1 | TRCN0000193988 | −1.94 |
| Smc3 | TRCN0000109006 | −1.92 |
| Nipbl | TRCN0000124037 | −1.87 |
| Smc1a | TRCN0000109032 | −1.86 |
| Ube2i | TRCN0000040839 | −1.86 |
| Ehmt1 | TRCN0000086071 | −1.86 |
| Suv39h2 | TRCN0000092815 | −1.85 |
| Ube2i | TRCN0000040841 | −1.85 |
| Stag2 | TRCN0000108979 | −1.84 |
| Ube2b | TRCN0000040869 | −1.82 |
| Wbscr22 | TRCN0000097427 | −1.79 |
| Setd7 | TRCN0000124111 | −1.78 |
| Setmar | TRCN0000120848 | −1.76 |
| Wbscr22 | TRCN0000097426 | −1.74 |
| Nipbl | TRCN0000124036 | −1.74 |

Table 3 shows a partial list of knockdowns that inhibit differentiation (increase in Oct4 staining). For these to be considered a hit they must have a Z-Score above 2.81 (greater than or equal to the Z-Score for the Tcf3 positive control) and phenotypically have formed good colonies (similar to the Tcf3 knockdown phenotype and indicative of cells that are not differentiating).

TABLE 3
Partial list of knockdowns that inhibit differentiation

| SYMBOL | CLONEID | Z-Score |
|---|---|---|
| Tcf3 | Tcf3_SpikeIn | 2.81 |
| Hira | TRCN0000081957 | 2.83 |
| Hmga1 | TRCN0000198788 | 2.91 |
| Arid1a | TRCN0000071394 | 2.98 |
| Cbx6 | TRCN0000096750 | 3.02 |
| Smc1b | TRCN0000109049 | 3.18 |
| Smarcb1 | TRCN0000087855 | 3.19 |
| Suv420h2 | TRCN0000039200 | 3.23 |
| Suv420h2 | TRCN0000039201 | 3.93 |
| Ankhd1 | TRCN0000193743 | 4.63 |

Applicants classified the genes listed in Tables 2 and 3 based on function and/or known presence in supramolecular complexes. Applicants identified the following categories of particular interest: methyltransferases, transcription factors, components of cohesion complex, chromatin assembly factors, chromatin associated factors, chromatin remodeling, sumoylation, ubiquitination, and heat shock. The classifications should not be interpreted as limiting. Certain genes may encode proteins with multiple activities and/or that participate in multiple different complexes. Table 1 (in FIG. 5) lists certain genes, their corresponding function/complex, associated phenotype with respect to Oct4 staining, and Z-score. The Z-Scores for the pluripotency and negative controls are shown as a reference.

A significant number of methyltransferases, in particular histone methyltransferases, were identified as regulators of pluripotency. Applicants observed that, with one exception (the H4K20 methyltransferase Suv420h2), shRNA that inhibit these methyltransferases resulted in decreased Oct4 staining. Applicants noted that the list of genes whose inhibition caused decreased Oct4 staining included three different H3K9 methyltransferases (Setdb1, Ehmt1, and Suv39h2) as well as Setd7 (also known as Set7/9). Applicants conclude that, in most cases, inhibiting histone methyltransferase activity (e.g., by inhibiting expression of histone methyltransferase(s)) promotes differentiation of pluripotent cells. Applicants' results point to a particularly important role for H3K9 methylation in regulating pluripotency/differentiation. In particular, inhibiting H3K9 methylation by inhibiting expression of any of four different H3K9 methyltransferases, resulted in decreased Oct4 staining, indicative of increased differentiation of the mES cells.

### Example 2

### Effect of Inhibiting H3K9 Methyltransferases on Generation of iPS Cells

Applicants next sought to determine the effect of inhibiting H3K9 methyltransferases on generation of iPS cells. For some experiments, Applicants used “secondary” mouse embryonic fibroblasts (MEFs) that express murine reprogramming factors Klf4, Sox2, and Oct4 under the control of a doxycycline (“dox”)-inducible promoter. These cells, which are referred to as “2nd KSO” cells for short, reprogram to form iPS cells at a low frequency upon treatment with doxycycline, as described in the literature. The cells contained an Oct4-neo transgene, thereby allowing use of G418 to select for cells that were reprogrammed to pluripotency (as evidenced by expression from the Oct4 promoter).

Applicants plated 2^{nd} KSO cells into individual wells of 6-well dishes (100,000 cells per well) in mES cell medium (3 ml) on day 0. On day 1, cells in individual wells were transfected with siRNA (Ambion) designed to inhibit expression of a gene encoding one of the following H3K9 methyltransferases: Ehmt1, Ehmt2, Suv39h1, Suv39h2, and Riz1. Two different siRNAs targeted to each of these genes were used (each well received a single siRNA sequence). The siRNA ID and the sequences of the siRNA sense and antisense strands are presented in the table below. Two different siRNAs (designated #1 and #2) targeted to each of these genes were used. It was subsequently noted that siRNA s82302 inhibited cell growth. Accordingly, results obtained using this siRNA must be disregarded. As negative controls, no siRNA and/or AM4611 (a non-targeting siRNA with minimal similarity to mammalian genes) were used. siRNA AM4620 (FAM) was used to monitor transfection efficiency. All siRNAs were purchased from Ambion/ABI. Typically results with the negative controls were very similar to one another. The siRNAs were used at a concentration of 50 nM in the medium. Typically, 2^{nd} KSO cells of passages ˜3-6 were used.

TABLE 4
siRNAs designed to inhibit H3K9 methyltransferase expression

| | | siRNA | siRNA | | |
|---|---|---|---|---|---|
| Gene | Function | # | ID | sequence SENSE | Sequence ANTISENSE |
| GLP/Ehmt1 | H3K9 | 1 | s95142 | GCACCUUUGUCUGCGAAUAtt | UAUUCGCAGACAAAGGUGCcc |
| | methyltransferase | 2 | s95141 | GAUCAAACCUGCUCGGAAAtt | UUUCCGAGCAGGUUUGAUCca |
| | (euc) | | | | |
| G9a/BAT8/Ehmt2 | H3K9 | 1 | 90229 | GGAGGAAGCUGAACUCUGGtt | CCAGAGUUCAGCUUCCUCCtt |
| | methyltransferase | 2 | s99719 | GAUUCUUACCUCUUCGAUUtt | AAUCGAAGAGGUAAGAAUCat |
| | (euc) | | | | |
| suv39h1 | H3K9 | 1 | 151927 | GGUGUACAACGUAUUCAUAtt | UAUGAAUACGUUGUACACCtg |
| | methyltransferase | 2 | 69566 | GGUCCUUUGUCUAUAUCAAtt | UUGAUAUAGACAAAGGACCtt |
| | (het) | | | | |
| suv39h2 | H3K9 | 1 | s82300 | GCUCACAUGUAAAUCGAUUtt | AAUCGAUUUACAUGUGAGCtt |
| | methyltransferase | 2 | s82302 | GUGUCGAUGUGGACCUGAAtt | UUCAGGUCCACAUCGACACct |
| | (het_testis sp.) | | | | |
| ESET/SetDB1 | H3K9 | 1 | s96548 | GGACUACAGUAUCAUGACAtt | UGUCAUGAUACUGUAGUCCca |
| | methyltransferase | 2 | s96547 | GGACGAUGCAGGAGAUAGAtt | UCUAUCUCCUGCAUCGUCCga |
| | | 3 | s96549 | GGAUGGGUGUCGGGAUAAAtt | UUUAUCCCGACACCCAUCCtt |
| PRDM2/Riz1 | H3K9 | 1 | s99829 | GAAUUUGCCUUCUUAUGCAtt | UGCAUAAGAAGGCAAAUUCtt |
| | methyltransferase | 2 | s99830 | GAGGAAUUCUAGUCCCGUAtt | UACGGGACUAGAAUUCCUCaa |

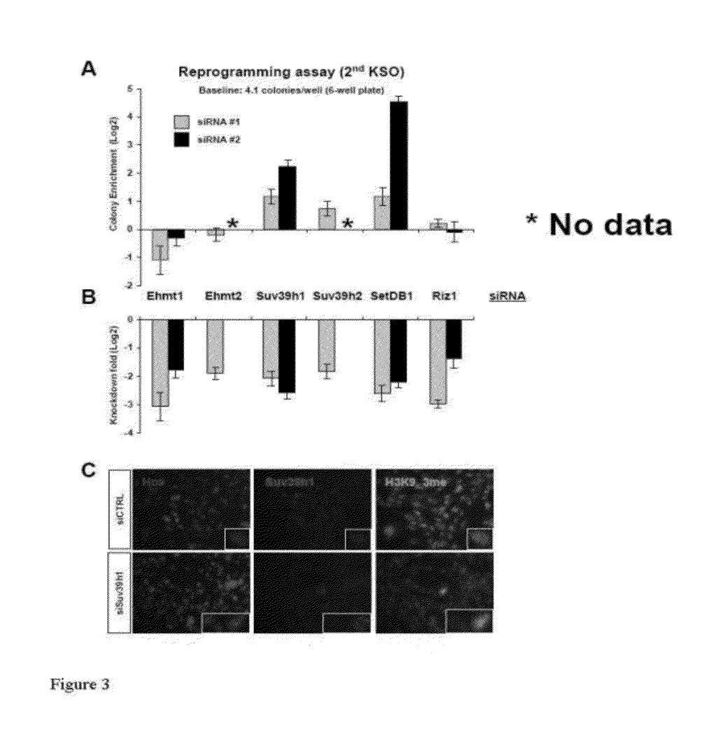

Cells were treated with dox to induce expression of the reprogramming factors. As a control, wells were treated with the same siRNAs but did not receive dox. Medium was changed on days 2, 5, 8, 11, 14, 17, and 20, with dox being included (except in afore-mentioned control wells). G418 was included in the medium at standard concentration starting at day 14 to select for reprogrammed cells. Colonies were counted on day 20. Wells that had been treated with dox and G418 but not with siRNA designed to inhibit H3K9 methyltransferase had an average of 4.1 colonies. As shown in FIG. 3A, certain siRNAs designed to inhibit Suv39h1, Suv39h2, or SetDB1 significantly increased the number of iPS cell colonies. The log (to the base 2) of the colony enrichment factor is shown on the y-axis. siRNA #2 against SetDB1 provided the most striking increase in reprogramming efficiency. FIG. 3B shows independent experiments designed to assess the extent of knockdown provided by the siRNAs.

Applicants confirmed by antibody staining that siRNA against Suv39h1 knocks down Suv39h1 protein levels and reduces H3K9 methylation by antibody staining. Results of co-stainings for Suv39h1 (Abcam, ab12405) and H3K9_{—}3me (Abeam, ab1186) are shown in FIG. 3C. “Hoe” indicates Hoechst dye staining. Treatment with siRNA inhibiting Suv39h1 resulted in a striking decrease in H3K9-3me versus treatment with a control siRNA.

### Example 3

### Inhibiting Suv39h1 and/or Suv39h2 Increases Reprogramming Efficiency

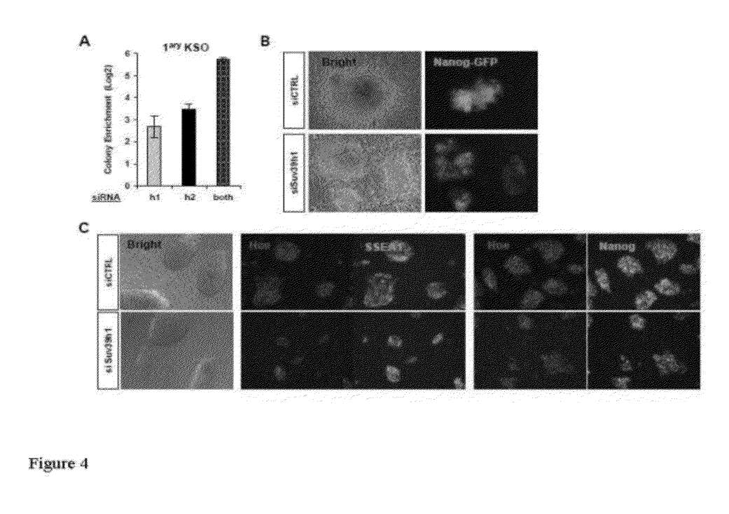

The experiments described in Example 2 were performed using a line of secondary MEFs in which expression of reprogramming factors was induced by dox treatment. In order to show that the increased reprogramming efficiency was not dependent on use of this system, Applicants performed “conventional” reprogramming experiments in which three dox-inducible retroviruses were used to express Klf4, Sox2, and Oct4. Primary MEFs harboring a Nanog-GFP transgene were used, thereby allowing identification of reprogrammed cells based on GFP expression. Cells were transfected in 10 cm plates with siRNA designed to inhibit Suv39h1, siRNA designed to inhibit Suv39h2, or a combination of the two siRNAs, and were maintained in culture. After 3 days, they were plated in 6-well plates at the same density that 2nd MEF. Colonies were counted 21 days after siRNA transfection and dox induction. As shown in FIG. 4A, inhibiting either Suv39h1 or Suv39h2 resulted in significant colony enrichment, while inhibiting both of these H3K9 methyltransferases resulted in still greater colony enrichment, indicating an additive effect. FIG. 4B shows colony appearance and GFP staining. FIG. 4C shows expression of the ES cell marker SSEA1, further confirming the identity of the reprogrammed cells. These experiments demonstrated that the increase in reprogramming efficiency achieved by inhibiting H3K9 methylation is not dependent on the use of secondary MEFs.

### Example 4

### Identification of Additional Reprogramming Agents

Secondary MEFs are cultured in the presence of an siRNA that inhibits histone methyltransferase and a candidate agent. In some embodiments the cells express only 2 of the following 3 reprogramming factors: Oct4, Klf4, and Sox2, Agents that enhance generating of reprogrammed cells (e.g., increase speed or efficiency of reprogramming) are identified. The process is repeated to identify agents capable of substituting for engineered expression of Klf4, Sox2, and/or Oct4 in reprogramming somatic cells.

### Example 5

### Use of Small Molecule Histone Methyltransferase Inhibitor in Reprogramming

Examples 2-4 are repeated, except that instead of using an siRNA that inhibits a histone methyltransferase, a small molecule inhibitor is used.

### REFERENCES

The following references (and references therein) relate to reprogramming somatic cells to pluripotency and describe certain reagents and methods of use in certain embodiments of the present invention.
Brambrink, T., Foreman, R., Welstead, G. G., Lengner, C. J., Wernig, M., Suh, H., and Jaenisch, R. (2008). Sequential expression of pluripotency markers during directreprogramming of mouse somatic cells. Cell Stem Cell 2, 151-159.
Cole, M. F., Johnstone, S. E., Newman, J. J., Kagey, M. H., and Young, R. A. (2008). Tcf3 is an integral component of the core regulatory circuitry of embryonic stem cells. Genes and Development 15; 22(6):746-55 (2008).
Hanna, J., Wernig, M., Markoulaki, S., Sun, C. W., Meissner, A., Cassady, J. P., Beard, C., Brambrink, T., Wu, L. C., Townes, T. M., and Jaenisch, R. (2007). Treatment of sickle cell anemia mouse model with iPS cells generated from autologous skin. Science 318, 1920-1923.
Hochedlinger, K., Yamada, Y., (2005) Cell. 121, 465-477.
Jaenisch, R., and Young, R. A. (2008). Stem cells, the molecular circuitry of pluripotency and nuclear reprogramming. Cell 132, 567-582.
Kim, J., Chu, J., Shen, X., Wang, J., and Orkin, S. H. (2008). An extended transcriptionalnetwork for pluripotency of embryonic stem cells. Cell 132, 1049-1061.
Meissner, A., Wernig, M., and Jaenisch, R. (2007). Direct reprogramming of genetically unmodified fibroblasts into pluripotent stem cells. Nat Biotechnol 25, 1177-1181.
Nakagawa, M., Koyanagi, M., Tanabe, K., Takahashi, K., Ichisaka, T., Aoi, T., Okita, K., Mochiduki, Y., Takizawa, N., and Yamanaka, S. (2008). Generation of inducedpluripotent stem cells without Myc from mouse and human fibroblasts. Nat Biotechnol 26, 101-106.
Okita, K., Ichisaka, T., and Yamanaka, S. (2007). Generation of germline-competentinduced pluripotent stem cells. Nature 448, 313-317.
Stadtfeld, M., Maherali, N., Breault, D. T., and Hochedlinger, K. (2008). Defining molecular cornerstones during fibroblast to iPS cell reprogramming in mouse. 2008 Mar. 6; 2(3):230-40. Epub 2008 Feb. 14.
Takahashi, K., Tanabe, K., Ohnuki, M., Narita, M., Ichisaka, T., Tomoda, K., and Yamanaka, S. (2007). Induction of pluripotent stem cells from adult human fibroblasts by defined factors. Cell 131, 861-872.
Takahashi, K., and Yamanaka, S. (2006). Induction of pluripotent stem cells from mouse embryonic and adult fibroblast cultures by defined factors. Cell 126, 663-676.
Tam, W. L., Lim, C. Y., Han, J., Zhang, J., Ang, Y. S., Ng, H. H., Yang, H., and Lim, B. (2008). Tcf3 Regulates Embryonic Stem Cell Pluripotency and Self-Renewal by the Transcriptional Control of Multiple Lineage Pathways. Stem Cells.
Wernig, M., Meissner, A., Cassady, J. P., and Jaenisch, R. (2008). c-Myc is dispensable for direct reprogramming of mouse fibroblasts. Cell Stem Cell 2, 10-12.
Yi, F., Pereira, L., and Merrill, B. J. (2008). Tcf3 Functions as a Steady State Limiter of Transcriptional Programs of Mouse Embryonic Stem Cell Self Renewal. Stem Cells.
Yu, J., Vodyanik, M. A., Smuga-Otto, K., Antosiewicz-Bourget, J., Frane, J. L., Tian, S., Nie, J., Jonsdottir, G. A., Ruotti, V., Stewart, R., et al. (2007). Induced pluripotent stem cell lines derived from human somatic cells. Science 318, 1917-1920.

The practice of the present invention will employ, unless otherwise indicated, conventional techniques of mouse genetics, developmental biology, cell biology, cell culture, molecular biology, transgenic biology, microbiology, recombinant DNA, and immunology, which are within the skill of the art. Such techniques are described in the literature. See, for example, Current Protocols in Cell Biology, ed. by Bonifacino, Dasso, Lippincott-Schwartz, Harford, and Yamada, John Wiley and Sons, Inc., New York, 1999; Manipulating the Mouse Embryos, A Laboratory Manual, 3^{rd} Ed., by Hogan et al., Cold Spring Contain Laboratory Press, Cold Spring Contain, New York, 2003; Gene Targeting: A Practical Approach, IRL Press at Oxford University Press, Oxford, 1993; and Gene Targeting Protocols, Human Press, Totowa, N.J., 2000. All patents, patent applications and references cited herein are incorporated in their entirety by reference.

One skilled in the art readily appreciates that the present invention is well adapted to carry out the objects and obtain the ends and advantages mentioned, as well as those inherent therein. The methods, systems and kits are representative of certain embodiments, are exemplary, and are not intended as limitations on the scope of the invention. Modifications therein and other uses will occur to those skilled in the art. These modifications are encompassed within the spirit of the invention and are defined by the scope of the claims. It will be readily apparent to a person skilled in the art that varying substitutions and modifications may be made to the invention disclosed herein without departing from the scope and spirit of the invention.

The articles “a” and “an” as used herein in the specification and in the claims, unless clearly indicated to the contrary, should be understood to include the plural referents. Claims or descriptions that include “or” between one or more members of a group are considered satisfied if one, more than one, or all of the group members are present in, employed in, or otherwise relevant to a given product or process unless indicated to the contrary or otherwise evident from the context. The invention includes embodiments in which exactly one member of the group is present in, employed in, or otherwise relevant to a given product or process. The invention also includes embodiments in which more than one, or all of the group members are present in, employed in, or otherwise relevant to a given product or process. Furthermore, it is to be understood that the invention encompasses all variations, combinations, and permutations in which one or more limitations, elements, clauses, descriptive terms, etc., from one or more of the listed claims is introduced into another claim dependent on the same base claim (or, as relevant, any other claim) unless otherwise indicated or unless it would be evident to one of ordinary skill in the art that a contradiction or inconsistency would arise. Where elements are presented as lists, e.g., in Markush group or similar format, it is to be understood that each subgroup of the elements is also disclosed, and any element(s) can be removed from the group. It should it be understood that, in general, where the invention, or aspects of the invention, is/are referred to as comprising particular elements, features, etc., certain embodiments of the invention or aspects of the invention consist, or consist essentially of, such elements, features, etc. For purposes of simplicity those embodiments have not in every case been specifically set forth herein. It should also be understood that any embodiment of the invention, can be explicitly excluded from the claims, regardless of whether the specific exclusion is recited in the specification. For example, any particular HMT or agent affecting histone methylation may be excluded.

Where ranges are given herein, the invention includes embodiments in which the endpoints are included, embodiments in which both endpoints are excluded, and embodiments in which one endpoint is included and the other is excluded. It should be assumed that both endpoints are included unless indicated otherwise. Furthermore, it is to be understood that unless otherwise indicated or otherwise evident from the context and understanding of one of ordinary skill in the art, values that are expressed as ranges can assume any specific value or subrange within the stated ranges in different embodiments of the invention, to the tenth of the unit of the lower limit of the range, unless the context clearly dictates otherwise. It is also understood that where a series of numerical values is stated herein, the invention includes embodiments that relate analogously to any intervening value or range defined by any two values in the series, and that the lowest value may be taken as a minimum and the greatest value may be taken as a maximum. Numerical values, as used herein, include values expressed as percentages. For any embodiment of the invention in which a numerical value is prefaced by “about” or “approximately”, the invention includes an embodiment in which the exact value is recited. For any embodiment of the invention in which a numerical value is not prefaced by “about” or “approximately”, the invention includes an embodiment in which the value is prefaced by “about” or “approximately”. “Approximately” or “about” is intended to encompass numbers that fall within a range of ±10% of a number, in some embodiments within ±5% of a number, in some embodiments within ±1%, in some embodiments within ±0.5% of a number, in some embodiments within ±0.1% of a number unless otherwise stated or otherwise evident from the context (except where such number would impermissibly exceed 100% of a possible value).

Where the claims or description recite a method, the invention provides compositions of use in practicing the method and further provides methods of making the compositions. Where the claims or description recite a composition, the invention provides methods of using the composition and methods of making the composition. Unless clearly indicated to the contrary, in any methods claimed herein that include more than one act, the order of the acts of the method is not limited to the order in which the acts of the method are recited, but the invention includes embodiments in which the order is so limited and embodiments in which the acts are performed during overlapping time intervals or over the same time interval.

1. A method of enhancing the reprogramming of mammalian cells comprising:
(a) contacting mammalian cells with an agent that inhibits histone methylation; and
(b) subjecting the cells to a reprogramming protocol so that at least some cells become reprogrammed to a desired cell state, wherein the agent enhances such reprogramming.

2. The method of claim 1, wherein the agent inhibits H3K9 methylation.

3. The method of claim 1, wherein the agent inhibits histone methyltransferase activity.

4. The method of claim 3, wherein inhibiting histone methyltransferase activity comprises inhibiting expression of a histone methyltransferase.

5. The method of claim 3, wherein the histone methyltransferase is an H3K9 methyltransferase.

6. The method of claim 3, wherein the histone methyltransferase is Suv39h1.

7. The method of claim 3, wherein the histone methyltransferase is Suv39h2.

8. The method of claim 3, wherein the histone methyltransferase is SetDB1.

9. The method of claim 3, wherein both Suv39h1 and Suv39h2 are inhibited.

10. The method of claim 1, wherein the agent is an siRNA or shRNA that inhibits expression of a histone methyltransferase.

11. The method of claim 10, wherein the histone methyltransferase is an H3K9 methyltransferase.

12. The method of claim 10, wherein the histone methyltransferase is Suv39h1.

13. The method of claim 10, wherein the histone methyltransferase is Suv39h2.

14. The method of claim 10, wherein the histone methyltransferase is SetDB1.

15. The method of claim 1, wherein the cells are differentiated cells, and reprogramming the cells comprises reprogramming the cells to a pluripotent state.

16. The method of claim 1, wherein the cells are iPS cells, and reprogramming the iPS cells comprises reprogramming the iPS cells to a desired cell type.

17. The method of claim 1, wherein the cells are differentiated cells of a first cell type, and the reprogramming protocol reprograms the cells to a second differentiated cell type.

18. The method of claim 1, wherein reprogramming efficiency is increased by at least a factor of 2.

19. The method of claim 1, wherein the cells are human cells.

20. The method of claim 1, wherein contacting the cells with the agent comprises culturing the cells in culture medium containing the agent.

21. The method of claim 1, wherein the cells are contacted with the agent for a limited period of time.

22. The method of claim 21, wherein the cells are contacted with the agent for between 1 and 10 days.

23. The method of claim 1, wherein the cells are modified to contain at least one reprogramming factor at levels greater than normally present in cells of that type.

24. The method of claim 23, wherein the cells comprise a nucleic acid construct that encodes the reprogramming factor, wherein the construct is not integrated into the cell genome.

25. The method of claim 1, wherein the cells are not genetically modified.

26. The method of claim 1, wherein the cells are not genetically modified to express c-Myc.

27. The method of claim 1, further comprising assessing whether the cells have become reprogrammed to the desired cell state.

28. The method of claim 1, further comprising separating cells that are reprogrammed to a desired state from cells that are not reprogrammed to a desired state.

29. The method of claim 1, further comprising administering the reprogrammed cells to a subject.

30. A method comprising:
(i) reprogramming somatic cells to a pluripotent state according to the method of claim 1; and
(ii) reprogramming the pluripotent cells to a desired, differentiated cell type.

31. A method comprising:
(i) reprogramming somatic cells to a pluripotent state; and
(ii) reprogramming the pluripotent cells to a desired, differentiated cell type according to the method of claim 1.

32. A method comprising:
(i) reprogramming somatic cells to a pluripotent state; and
(ii) reprogramming the pluripotent cells to a desired, differentiated cell type, wherein step (i) and step (ii) are performed according to the method of claim 1.

33. The method of claim 1, wherein the reprogramming protocol comprises inducing expression of at least one reprogramming factor in the cells.

34. A method of treating an individual in need thereof comprising:
(a) obtaining somatic cells from the individual;
(b) reprogramming at least some of the somatic cells according to the method of claim 1; and
(c) administering at least some of the reprogrammed cells to the individual.

35. The method of claim 34, wherein the method further comprises separating cells that are reprogrammed to a desired state from cells that are not reprogrammed to a desired state.

36. The method of claim 34, wherein the individual is a human.

37. A composition comprising (i) a non-pluripotent somatic mammalian cell that comprises an introduced reprogramming factor; and (ii) an agent that inhibits histone methylation.

38. The composition of claim 37, wherein the reprogramming factor is Oct4.

39. The composition of claim 37, wherein the agent is an siRNA.

40. The composition of claim 37, wherein the somatic cell is not genetically modified.

41. The composition of claim 37, wherein the somatic cell does not contain exogenously introduced c-Myc at levels greater than normally present in somatic cells of that type.

42. A composition comprising (i) an iPS cell; and (ii) an agent that inhibits histone methylation.

43. The composition of claim 42, wherein the agent is an siRNA.

44. The composition of claim 42, wherein the iPS cell is not genetically modified.

45. A method of identifying an agent useful for modulating the reprogramming of mammalian cells comprising:
(a) maintaining mammalian cells in culture in the presence of a candidate agent under conditions in which histone methylation is inhibited in the cells, wherein the mammalian cells are cells of a first cell type; and
(b) determining, after a suitable time period, whether cells having one or more characteristics of a second cell type different from the first cell type are present in the culture, wherein the candidate agent is identified as being useful for modulating the reprogramming of mammalian cells if cells or cell colonies having one or more characteristics of the second cell type are present in amounts different than would be expected had the cells of the first cell type been cultured under identical conditions in the absence of the candidate agent.

46. The method of claim 45, wherein the cells of the first cell type are somatic cells.

47. The method of claim 45, wherein the cells of the first cell type are somatic cells and cells of the second cell type are ES cells.

48. The method of claim 45, wherein the cells of the first cell type are terminally differentiated cells.

49. The method of claim 45, wherein the cells of the first cell type are ES cells.

50. The method of claim 45, wherein the cells of the first cell type are iPS cells.

51. The method of claim 45, wherein the cells of the first cell type are iPS cells and cells of the second cell type are terminally differentiated cells.

52. The method of claim 45, wherein the cells contain at least one introduced reprogramming factor.

53. The method of claim 45, wherein the candidate agent is a small molecule.

54. The method of claim 45, wherein H3K9 methylation is inhibited.

55. The method of claim 45, wherein histone methylation is inhibited by contacting the cells with an siRNA that inhibits expression of a histone methyltransferase.

56. The method of claim 45, wherein cells of the first cell type are non-pluripotent somatic cells, cells of the second cell type are pluripotent cells, wherein the candidate agent is identified as being useful for enhancing the reprogramming of non-pluripotent mammalian somatic cells to a pluripotent state if cells or cell colonies having one or more characteristics of ES cells or ES cell colonies are present at levels greater than would be expected had the cells been cultured under identical conditions in the absence of the candidate agent.

57. A method of identifying an agent useful for modulating the reprogramming of mammalian cells comprising:
(a) maintaining mammalian ES or iPS cells in culture in the presence of a candidate agent; and
(b) assessing expression of an endogenous pluripotency gene by the cells, wherein the agent is identified as useful for modulating the reprogramming of mammalian cells if expression of the endogenous pluripotency gene is increased or decreased relative to the level of expression of said gene that would exist in the absence of the candidate agent.

58. The method of claim 57, wherein the agent is identified as useful for reprogramming mammalian somatic cells to a less differentiated state if expression is increased.

59. The method of claim 57, wherein the agent is identified as useful for reprogramming mammalian somatic cells to a more differentiated state if expression is decreased.

60. The method of claim 57, wherein the pluripotency gene is Oct4.

61. A method of identifying a gene whose inhibition modulates the reprogramming of mammalian cells comprising:
(a) providing mammalian ES or iPS cells in culture; and
(b) inhibiting expression of an endogenous candidate gene by the ES or iPS cells; and
(c) assessing expression of an endogenous pluripotency gene by the cells, wherein the endogenous candidate gene is identified as one whose inhibition modulates the reprogramming of mammalian cells if expression of the endogenous pluripotency gene is increased or decreased relative to the level of expression of said gene that would exist in ES or iPS cells in which expression of the candidate gene is not inhibited.

62. The method of claim 61, wherein the gene is identified as one whose inhibition promotes reprogramming of mammalian somatic cells to a less differentiated state if expression of the endogenous pluripotency gene is increased.

63. The method of claim 61, wherein the gene is identified as one whose inhibition promotes reprogramming of mammalian cells to a more differentiated state if expression of the endogenous pluripotency gene is decreased.

64. The method of claim 61, wherein the pluripotency gene is Oct4.

65. The method of claim 61, wherein expression of the endogenous candidate gene is inhibited by RNAi.

66. A method of identifying an agent useful for modulating reprogramming of mammalian cells, the method comprising identifying an agent that inhibits expression or activity of a gene identified according to the method of claim 61.


SEQUENCE LISTING (26 sequences)

SEQ ID NO: 1 - DNA, 21 bp, Artificial
  siRNA sequence
gcaccuuugucugcgaauatt

SEQ ID NO: 2 - DNA, 21 bp, Artificial
  siRNA sequence
gaucaaaccugcucggaaatt

SEQ ID NO: 3 - DNA, 21 bp, Artificial
  siRNA sequence
ggaggaagcugaacucuggtt

SEQ ID NO: 4 - DNA, 21 bp, Artificial
  siRNA sequence
gauucuuaccucuucgauutt

SEQ ID NO: 5 - DNA, 21 bp, Artificial
  siRNA sequence
gguguacaacguauucauatt

SEQ ID NO: 6 - DNA, 21 bp, Artificial
  siRNA sequence
gguccuuugucuauaucaatt

SEQ ID NO: 7 - DNA, 21 bp, Artificial
  siRNA sequence
gcucacauguaaaucgauutt

SEQ ID NO: 8 - DNA, 21 bp, Artificial
  siRNA sequence
gugucgauguggaccugaatt

SEQ ID NO: 9 - DNA, 21 bp, Artificial
  siRNA sequence
ggacuacaguaucaugacatt

SEQ ID NO: 10 - DNA, 21 bp, Artificial
  siRNA sequence
ggacgaugcaggagauagatt

SEQ ID NO: 11 - DNA, 21 bp, Artificial
  siRNA sequence
ggaugggugucgggauaaatt

SEQ ID NO: 12 - DNA, 21 bp, Artificial
  siRNA sequence
gaauuugccuucuuaugcatt

SEQ ID NO: 13 - DNA, 21 bp, Artificial
  siRNA sequence
gaggaauucuagucccguatt

SEQ ID NO: 14 - DNA, 21 bp, Artificial
  siRNA sequence
uauucgcagacaaaggugccc

SEQ ID NO: 15 - DNA, 21 bp, Artificial
  siRNA sequence
uuuccgagcagguuugaucca

SEQ ID NO: 16 - DNA, 21 bp, Artificial
  siRNA sequence
ccagaguucagcuuccucctt

SEQ ID NO: 17 - DNA, 21 bp, Artificial
  siRNA sequence
aaucgaagagguaagaaucat

SEQ ID NO: 18 - DNA, 21 bp, Artificial
  siRNA sequence
uaugaauacguuguacacctg

SEQ ID NO: 19 - DNA, 21 bp, Artificial
  siRNA sequence
uugauauagacaaaggacctt

SEQ ID NO: 20 - DNA, 21 bp, Artificial
  siRNA sequence
aaucgauuuacaugugagctt

SEQ ID NO: 21 - DNA, 21 bp, Artificial
  siRNA sequence
uucagguccacaucgacacct

SEQ ID NO: 22 - DNA, 21 bp, Artificial
  siRNA sequence
ugucaugauacuguaguccca

SEQ ID NO: 23 - DNA, 21 bp, Artificial
  siRNA sequence
ucuaucuccugcaucguccga

SEQ ID NO: 24 - DNA, 21 bp, Artificial
  siRNA sequence
uuuaucccgacacccaucctt

SEQ ID NO: 25 - DNA, 21 bp, Artificial
  siRNA sequence
ugcauaagaaggcaaauuctt

SEQ ID NO: 26 - DNA, 21 bp, Artificial
  siRNA sequence
uacgggacuagaauuccucaa